(19) United States
(12) Patent Application Publication
(10) Pub. No.: US 2024/0182821 A1
(43) Pub. Date: Jun. 6, 2024

(54) TREATMENT COMPOSITION WITH PERFUME-CONTAINING DELIVERY PARTICLES

(71) Applicant: The Procter & Gamble Company, Cincinnati, OH (US)

(72) Inventors: AMANDA KISER JUKES, Brussels (BE); RAFAEL TRUJILLO, MASON, OH (US); CONNY ERNA ALICE JOOS, BUGGENHOUT (BE); PRAKASH J. MADHAV, MAINEVILLE, OH (US); JOHAN SMETS, LUBBEEK (BE); SUSANA FERNANDEZ PRIETO, BENICARLO (ES); MATTIA COLLU, SAINT-GILLES (BE); LINSHENG FENG, MENASHA, WI (US)

(21) Appl. No.: 18/522,443

(22) Filed: Nov. 29, 2023

Related U.S. Application Data
(60) Provisional application No. 63/429,186, filed on Dec. 1, 2022.

(51) Int. Cl. C11D 3/50; A61K 8/11; A61K 8/73; A61Q 5/00; A61Q 15/00; C11D 3/22

(52) U.S. Cl. CPC C11D 3/505; A61K 8/11; A61K 8/736; A61Q 5/00; A61Q 15/00; C11D 3/227; A61K 2800/654; C11D 2111/14

(57) ABSTRACT

A treatment composition that includes a treatment adjunct and a population of core/shell delivery particles, where the shell includes a polymeric material formed from a cross-linked amine-containing biopolymer, and where the core includes a fragrance material that includes ester-containing perfume raw materials. Related methods of making and using such compositions.

### FIELD OF THE INVENTION

The present disclosure relates to a treatment composition that includes a treatment adjunct and a population of core/shell delivery particles, where the shell includes a polymeric material formed from a cross-linked amine-containing biopolymer, and where the core includes a fragrance material that includes ester-containing perfume raw materials. The present disclosure also relates to related methods of making and using such compositions.

### BACKGROUND OF THE INVENTION

Delivery particles, particularly core/shell delivery particles, are a convenient way to deliver benefit agents in treatment compositions such as laundry products. For environmental reasons, it may be desirable to use delivery particles that have a shell made from naturally-derived and/or biodegradable materials. Useful shell-making materials often include primary amine groups, as such materials readily react with a number of cross-linkers, such as polyisocyanates, to form a suitable polymeric material.

That being said, when encapsulating perfume in core-shell delivery particles, certain shell materials can interact with certain perfume raw materials, resulting in a relatively poor-performing capsules. For example, when making particles that have polymeric shells made from materials that contain primary amines (such as chitosan), perfume raw materials that contain aldehyde groups can react with the amine groups, which may result in relatively weak and/or leaky capsules. In particular, encapsulation of aldehyde-containing perfumes in polyurea shells is known to be challenging.

There is a need for improved treatment compositions that include perfume-containing delivery particles, preferably particles that are made, at least in part, from naturally-derived or biodegradable materials.

### SUMMARY OF THE INVENTION

The present disclosure relates to treatment compositions that include delivery particles, where the particles include ester-containing perfume raw materials in the core.

For example, the present disclosure relates to a treatment composition that includes: a treatment adjunct, and a population of delivery particles, where the delivery particles include a core and a shell surrounding the core, where the shell comprises a polymeric material, where the polymeric material includes a reaction product of a biopolymer and a cross-linking agent, where the biopolymer includes primary amine groups, where the core includes a fragrance material, where the fragrance material includes: (a) at least about 30%, by weight of the fragrance material, of ester-containing perfume raw materials (“PRMs”), and (b) at least about 0.5wt %, by weight of the fragrance material, aldehyde-containing PRMs.

The present disclosure also relates to a treatment composition that includes: a treatment adjunct, and a population of delivery particles, where the delivery particles includes a core and a shell surrounding the core, where the shell includes a polymeric material, where the polymeric material includes a reaction product of a biopolymer and a cross-linking agent, where the biopolymer includes primary amine groups, where the core comprises a fragrance material, where the fragrance material includes one or more perfume raw materials (“PRMs”), and where the fragrance material is characterized by one or more of the following: (a) as having an S-ESTER value of at least about 5, wherein the S-ESTER value of the fragrance material is calculated as the weight average S-ESTER values of the perfume raw materials; and/or (b) as including at least about 30%, by weight of the fragrance materials, of perfume raw materials having S-ESTER values of 13 or greater.

The present disclosure also relates to a method of treating a surface, the method including the step of: contacting the surface, preferably a fabric, with a treatment composition according to the present disclosure.

The present disclosure also relates to a method of making a treatment composition, the method including the steps of: providing a base composition, where the base composition includes a treatment adjunct, and combining a population of delivery particles with the base composition, where the delivery particles include a core and a shell surrounding the core, where the shell includes a polymeric material, where the polymeric material includes a reaction product of a biopolymer and a cross-linking agent, where the biopolymer include primary amine groups, where the core includes a fragrance material, where the fragrance material includes: (a) at least about 30%, by weight of the fragrance material, of ester-containing perfume raw materials (“PRMs”), and (b) at least about 0.5wt %, by weight of the fragrance material, aldehyde-containing PRMs. Additionally or alternatively, the fragrance material may be characterized by one or more of the following: (a) as having an S-ESTER value of at least about 5, wherein the S-ESTER value of the fragrance material is calculated as the weight average S-ESTER values of the perfume raw materials; and/or (b) as including at least about 30%, by weight of the fragrance materials, of perfume raw materials having S-ESTER values of 13 or greater.

### DETAILED DESCRIPTION OF THE INVENTION

The present disclosure relates to treatment compositions that include perfume-containing delivery particles having shells made, at least in part, from biopolymers that include primary amine groups. It has been found that careful selection of the fragrance material to be encapsulated in the core can result in improved freshness performance. In particular, selecting minimum certain amounts of ester-containing perfume raw materials (“PRMs”) has been found to be advantageous.

Without wishing to be bound by theory, it is believed that the ester-containing PRMs create hydrogen-bonding or dipole moments that “engage” with the amine groups of the amine-containing biopolymer, thereby reducing the reaction of the “available” or “free” amine groups with the aldehydic PRMs and/or the ketone-containing PRMs. Although the groups may temporarily “engage,” it is believed that the temperatures at which such delivery particles are typically made (e.g., 50-95° C.) do not favor reactions between the amine groups and the ester groups. Furthermore, it is believed that an amine/ester reaction is kinetically disfavored compared to amine reactions with isocyanates (e.g., from a cross-linking agent) or an aldehyde (e.g., from another PRM), even though the hydrogen-bonding capacity of the ester groups is relatively strong, which is reflected in the S-ESTER descriptor described herein.

To further improve the shell-formation and resulting performance of the capsules, it may be preferred to limit the amount of aldehyde-, ketone-, and hydroxyl-containing PRMs in the fragrance to be encapsulated.

The fragrances, delivery particles, treatment compositions, and related methods of the present disclosure are discussed in more detail below.

As used herein, the articles “a” and “an” when used in a claim, are understood to mean one or more of what is claimed or described. As used herein, the terms “include,” “includes,” and “including” are meant to be non-limiting. The compositions of the present disclosure can comprise, consist essentially of, or consist of, the components of the present disclosure.

The terms “substantially free of” or “substantially free from” may be used herein. This means that the indicated material is at the very minimum not deliberately added to the composition to form part of it, or, preferably, is not present at analytically detectable levels. It is meant to include compositions whereby the indicated material is present only as an impurity in one of the other materials deliberately included. The indicated material may be present, if at all, at a level of less than 1%, or less than 0.1%, or less than 0.01%, or even 0%, by weight of the composition.

As used herein “consumer product,” means baby care, beauty care, fabric & home care, family care, feminine care, and/or health care products or devices intended to be used or consumed in the form in which it is sold, and not intended for subsequent commercial manufacture or modification. Such products include but are not limited to diapers, bibs, wipes; products for and/or methods relating to treating human hair, including bleaching, coloring, dyeing, conditioning, shampooing, styling; deodorants and antiperspirants; personal cleansing; skin care including application of creams, lotions, and other topically applied products for consumer use; and shaving products, products for and/or methods relating to treating fabrics, hard surfaces and any other surfaces in the area of fabric and home care, including: air care, car care, dishwashing, fabric conditioning (including softening), laundry detergency, laundry and rinse additive and/or care, hard surface cleaning and/or treatment, and other cleaning for consumer or institutional use; products and/or methods relating to bath tissue, facial tissue, paper handkerchiefs, and/or paper towels; tampons, feminine napkins; adult incontinence products; products and/or methods relating to oral care including toothpastes, tooth gels, tooth rinses, denture adhesives, tooth whitening; over-the-counter health care including cough and cold remedies; pest control products; and water purification.

As used herein the phrase “fabric care composition” includes compositions and formulations designed for treating fabric. Such compositions include but are not limited to, laundry cleaning compositions and detergents, fabric softening compositions, fabric enhancing compositions, fabric freshening compositions, laundry prewash, laundry pretreat, laundry additives, spray products, dry cleaning agent or composition, laundry rinse additive, wash additive, post-rinse fabric treatment, ironing aid, unit dose formulation, delayed delivery formulation, detergent contained on or in a porous substrate or nonwoven sheet, and other suitable forms that may be apparent to one skilled in the art in view of the teachings herein. Such compositions may be used as a pre-laundering treatment, a post-laundering treatment, or may be added during the rinse or wash cycle of the laundering operation.

As used herein, “delivery particles,” “particles,” “encapsulates,” “microcapsules,” and “capsules” are used interchangeably, unless indicated otherwise. As used herein, these terms typically refer to core/shell delivery particles.

Unless otherwise noted, all component or composition levels are in reference to the active portion of that component or composition, and are exclusive of impurities, for example, residual solvents or by-products, which may be present in commercially available sources of such components or compositions.

All temperatures herein are in degrees Celsius (C) unless otherwise indicated. Unless otherwise specified, all measurements herein are conducted at 20° C. and under the atmospheric pressure.

In all embodiments of the present disclosure, all percentages are by weight of the total composition, unless specifically stated otherwise. All ratios are weight ratios, unless specifically stated otherwise.

It should be understood that every maximum numerical limitation given throughout this specification includes every lower numerical limitation, as if such lower numerical limitations were expressly written herein. Every minimum numerical limitation given throughout this specification will include every higher numerical limitation, as if such higher numerical limitations were expressly written herein. Every numerical range given throughout this specification will include every narrower numerical range that falls within such broader numerical range, as if such narrower numerical ranges were all expressly written herein.

### Treatment Composition

The present disclosure relates to treatment compositions (or simply “compositions” as used herein). The compositions of the present disclosure may comprise a population of delivery particles and a treatment adjunct, each described in more detail below. The treatment compositions may be useful in the methods of treating surfaces, such as fabrics, described herein.

The treatment composition is preferably a consumer product composition. The consumer products compositions of the present disclosure may be useful in baby care, beauty care, fabric care, home care, family care, feminine care, and/or health care applications. The consumer product compositions may be useful for treating a surface, such as fabric, hair, or skin. The consumer product compositions may be intended to be used or consumed in the form in which it is sold. The consumer product compositions of the present disclosure are typically not intended for subsequent commercial manufacture or modification.

The consumer product composition may preferably be a fabric care composition, a hard surface cleaner composition, a dish care composition, a hair care composition (such as shampoo or conditioner), a body cleansing composition, or a mixture thereof, preferably a fabric care composition.

The consumer product composition may be a fabric care composition, such as a laundry detergent composition (including a heavy-duty liquid washing detergent or a unit dose article), a fabric conditioning composition (including a liquid fabric softening and/or enhancing composition), a laundry additive, a fabric pre-treat composition (including a spray, a pourable liquid, or a spray), a fabric refresher composition (including a spray), or a mixture thereof. The treatment composition is preferably a fabric conditioning composition, even more preferably a liquid fabric conditioning composition.

The composition may be a beauty care composition, such as a hair treatment product (including shampoo and/or conditioner), a skin care product (including a cream, lotion, or other topically applied product for consumer use), a shave care product (including a shaving lotion, foam, or pre- or post-shave treatment), personal cleansing product (including a liquid body wash, a liquid hand soap, and/or a bar soap), a deodorant and/or antiperspirant, or mixtures thereof.

The composition may be a home care composition, such as an air care, car care, dishwashing, hard surface cleaning and/or treatment, and other cleaning compositions for consumer or institutional use.

The treatment composition may be in the form of a liquid composition, a granular composition, a hydrocolloid, a single-compartment pouch, a multi-compartment pouch, a dissolvable sheet, a pastille or bead, a fibrous article, a tablet, a stick, a bar, a flake, a foam/mousse, a non-woven sheet, or a mixture thereof.

The treatment composition may be in the form of a liquid. The liquid composition may preferably include from about 50% to about 97%, preferably from about 60% to about 96%, more preferably from about 70% to about 95%, or even from about 80% to about 95%, by weight of the fabric treatment composition, of water. The liquid composition may be a liquid fabric conditioner. The liquid may be packaged in a pourable bottle. The liquid may be packaged in an aerosol can or other spray bottle. Suitable containers are described in more detail below.

The treatment composition may be in the form of a solid. The composition may be in the form of a bead or pastille, which may be pastilled from a liquid melt. The composition may be an extruded product. The treatment composition may be in the form of a powder or granules.

The composition may be in the form of a unitized dose article, such as a tablet, a pouch, a sheet, or a fibrous article. Such pouches typically include a water-soluble film, such as a polyvinyl alcohol water-soluble film, that at least partially encapsulates a composition. Suitable films are available from MonoSol, LLC (Indiana, USA). The composition can be encapsulated in a single or multi-compartment pouch. A multi-compartment pouch may have at least two, at least three, or at least four compartments. A multi-compartmented pouch may include compartments that are side-by-side and/or superposed. The composition contained in the pouch or compartments thereof may be liquid, solid (such as powders), or combinations thereof. Pouched compositions may have relatively low amounts of water, for example less than about 20%, or less than about 15%, or less than about 12%, or less than about 10%, or less than about 8%, by weight of the detergent composition, of water.

The treatment composition may be in the form of a spray and may be dispensed, for example, from a bottle via a trigger sprayer and/or an aerosol container with a valve.

The treatment composition may have a viscosity of from 1 to 1500 centipoises (1-1500 mPa*s), from 100 to 1000 centipoises (100-1000 mPa*s), or from 200 to 500 centipoises (200-500 mPa*s) at 20 s⁻¹ and 21° C.

The treatment compositions of the present disclosure may be characterized by a pH of from about 2 to about 12, or from about 2 to about 8.5, or from about 2 to about 7, or from about 2 to about 5. The treatment compositions of the present disclosure may have a pH of from about 2 to about 4, preferably a pH of from about 2 to about 3.7, more preferably a pH from about 2 to about 3.5, preferably in the form of an aqueous liquid. It is believed that such PH levels facilitate stability of the quaternary ammonium ester compound, when present. On the other hand, detergent compositions are typically characterized by a pH of from about 7 to about 12, preferably from about 7.5 to about 11. The pH of a composition is determined by dissolving/dispersing the composition in deionized water to form a solution at 10% concentration, at about 20° C.

Additional components and/or features of the compositions are discussed in more detail below.

### Population of Delivery Particles

The treatment compositions of the present disclosure comprise a population of delivery particles. The delivery particles comprise a core and a shell surrounding the core. The core comprises a fragrance material, and optionally a partitioning modifier. The core can be a liquid or a solid, preferably a liquid, at room temperature.

The treatment composition may comprise from about 0.05% to about 20%, or from about 0.05% to about 10%, or from about 0.1% to about 5%, or from about 0.2% to about 2%, by weight of the composition, of delivery particles. The composition may comprise a sufficient amount of delivery particles to provide from about 0.05% to about 10%, or from about 0.1% to about 5%, or from about 0.1% to about 2%, by weight of the composition, of the encapsulated benefit agent, which may preferably be perfume raw materials, to the composition. When discussing herein the amount or weight percentage of the delivery particles, it is meant the sum of the wall material and the core material.

The population of delivery particles according to the present disclosure may be characterized by a volume-weighted median particle size from about 1 to about 100 microns, preferably from about 10 to about 100 microns, preferably from about 15 to about 50 microns, more preferably from about 20 to about 40 microns, even more preferably from about 25 to about 35 microns. For certain compositions, it may be preferred that the population of delivery particles is characterized by a volume-weighted median particle size from about 1 to about 50 microns, preferably from about 5 to about 20 microns, more preferably from about 10 to about 15 microns. Different particle sizes are obtainable by controlling droplet size during emulsification.

The delivery particles may be characterized by a ratio of core to shell up to 99:1, or even 99.5:0.5, on the basis of weight. The shell may be present at a level of from about 1% to about 25%, preferably from about 1% to about 20%, preferably from about 1% to 15%, more preferably from about 5% to about 15%, even more preferably from about 10% to about 15%, even more preferably from about 10% to about 12%, by weight of the delivery particle. The shell may be present at a level of least 1%, preferably at least 3%, more preferably at least 5% by weight of the delivery particle. The shell may be present at a level of up to about 25%, preferably up to about 20%, preferably up to about 15%, more preferably up to about 12%, by weight of the delivery particle.

The delivery particles may be cationic in nature, preferably cationic at a pH of 4.5. The delivery particles may be characterized by a zeta potential of at least 15 millivolts (mV) at a pH of 4.5. The delivery particles can be fashioned to have a zeta potential of at least 15 millivolts (mV) at a pH of 4.5, or even at least 40 mV at a pH of 4.5, or even at least 60 mV at a pH of 4.5. Delivery particles prepared with chitosan typically exhibit positive zeta potentials. Such capsules have improved deposition efficiency on fabrics. At higher pH, the particles may be able to be made nonionic or anionic.

The delivery particles of the present disclosure comprise a shell surrounding a core. (As used herein, “shell” and “wall” are used interchangeably with regard to the delivery particles, unless indicated otherwise.) The shell comprises a polymeric material. The polymeric material is the reaction product of a biopolymer and a cross-linking agent.

The biopolymer typically comprises primary amine groups. The primary amine groups react with the cross-linking agent to form the polymeric material, which may be described as a cross-linked biopolymer.

The biopolymer may preferably be selected from the group consisting of a polysaccharide, a protein, a nucleic acid, derivatives thereof, and combinations thereof. Preferably, the biopolymer is selected from the group consisting of:
(a) a polysaccharide selected from the group consisting of chitosan, amine-modified starch, amine-modified dextran, amine-modified maltodextrin, amine-modified dextrin, amine-modified cellulose, amine-modified hemicellulose, chitin, amine modified alginate, amine modified lignin, amine modified gum, amine modified pectin, amine modified agar, melanin, silk fibronin, derivatives thereof, and combinations thereof;
(b) a protein selected from the group consisting of gelatin (e.g., pig-derived gelatin), collagen, casein, sericin, fibroin, whey protein, pea protein, zein, soy protein, plant storage protein (plant protein isolate, plant protein concentrate), gluten, peptide, actin, derivatives thereof, and combinations thereof;
(c) a nucleic acid selected from the group consisting of polynucleotides, RNA, DNA, derivatives thereof, and combinations thereof; and
(d) combinations thereof.

Amine-containing polysaccharides may be preferred, for example due to convenient availability, biodegradability, and/or performance reasons. A particularly preferred material is chitosan. Thus, the biopolymer may preferably be chitosan, a derivative thereof, or a combination thereof. Preferably, the biopolymer is acid-treated chitosan, a derivative thereof, or a combination thereof.

The chitosan may preferably be acid-treated chitosan. For example, chitosan (which, prior to acid treatment, may be referred to as raw chitosan or parent chitosan) may preferably be treated at a pH of 6.5 or less with an acid for at least one hour, preferably from about one hour to about three hours, or for a period of time required to obtain a chitosan solution viscosity of not more than about 1500 cps of the acid-treated chitosan, or even not more than 500 cps, at a temperature of from about 25° C. to about 99° C., preferably from about 75° C. to about 95° C. The acid may be selected from a strong acid (such as hydrochloric acid), an organic acid (such as formic acid or acetic acid), or a mixture thereof. The chitosan may preferably be acid-treated at a pH of from 2 to 6.5, preferably a pH of from about 3 to about 6, or even from a pH of from 4 to 6.

The biopolymer, preferably chitosan, more preferably acid-treated chitosan, may preferably be characterized by a molecular weight of from about 1 kDa to about 1000 kDa, preferably from about 50 kDa to about 600 kDa, more preferably from about 100 kDa to about 500 kDa, even more preferably from about 100 kDa to about 300 kDa, even more preferably from about 100 kDa to about 200 kDa. Without wishing to be bound by theory, it is believed that biopolymers characterized by a relatively low molecular weight are less effective at forming suitable delivery particles, while those having relatively high molecular weights tend to be difficult to process. The method used to determine the chitosan's molecular weight and related parameters is provided in the Test Methods section below and uses gel permeation chromatograph with multi-angle light scatter and refractive index detection (GPC-MALS/RI) techniques.

The chitosan may be characterized by a degree of deacetylation of at least 50%, preferably from about 50% to about 99%, more preferably from about 75% to about 90%, even more preferably from about 80% to about 85%. The degree of deacetylation can affect the solubility of the chitosan, which in turn can affect its reactivity or behavior in the process of forming the particle shells. For example, a degree of deacetylation that is too low (e.g., below 50%) results in chitosan that is relatively insoluble and relatively unreactive. A degree of deacetylation that is relatively high can result in chitosan that is very soluble, resulting in relatively little of it traveling to the oil/water interface during shell formation.

The chitosan, when present, may comprise anionically modified chitosan, cationically modified chitosan, or a combination thereof. Modifying the chitosan in an anionic and/or cationic fashion can change the character of the shell of the delivery particle, for example, by changing the surface charge and/or zeta potential, which can affect the deposition efficiency and/or formulation compatibility of the particles.

As mentioned above, the shell is a polymeric material that is the reaction product of the biopolymer chitosan and a cross-linking agent. The cross-linking material is preferably a material selected from the group consisting of a polyisocyanate, a polyacrylate, a poly(meth)acrylate, a polyisothiocyanate, an aldehyde, an epoxy compound, a polyphenol, a carbonyl halide, an aziridine, and combinations thereof. The cross-linking agent is more preferably selected from the group consisting of a polyisocyanate, an epoxy compound, a bifunctional aldehyde, and combinations thereof.

The cross-linking agent is preferably a polyisocyanate. It is believed that such materials favorable react with the amine groups of the biopolymer to form effective, cross-linked polymeric walls. When the cross-linking agent is a polyisocyanate, the polymeric material of the shells may be understood to comprise a polyurea resin. The polyurea resin may comprise the reaction product of a polyisocyanate and a chitosan.

The polyisocyanate material useful in the present disclosure is to be understood for purposes hereof as isocyanate monomer, isocyanate oligomer, isocyanate prepolymer, or dimer or trimer of an aliphatic or aromatic isocyanate. By “polyisocyanate,” it is intended to mean a material or compound that includes two or more isocyanate moieties. All such monomers, prepolymers, oligomers, or dimers or trimers of aliphatic or aromatic isocyanates are intended encompassed by the term “polyisocyanate” herein. The polyisocyanates useful in the present disclosure comprise isocyanate monomers, oligomers or prepolymers, or dimers or trimers thereof, having at least two isocyanate groups. Preferred cross-linking can be achieved with polyisocyanates having at least three functional groups.

Aromatic polyisocyanates may be preferred; however, aliphatic polyisocyanates and blends thereof may be useful. Aliphatic polyisocyanate is understood as a polyisocyanate which does not comprise any aromatic moiety. Aromatic polyisocyanate is understood as a polyisocyanate which comprises at least one aromatic moiety. The cross-linking agent may comprise a mixture of an aromatic polyisocyanate and an aliphatic polyisocyanate.

The polyisocyanate, when aromatic, can be, but is not limited to, methylene diphenyl isocyanate, toluene diisocyanate, tetramethylxylidene diisocyanate, polyisocyanurate of toluene diisocyanate (commercially available from Bayer under the tradename Desmodur® RC), trimethylol propane-adduct of toluene diisocyanate (commercially available from Bayer under the tradename Desmodur® L75), naphthalene-1,5-diisocyanate, and phenylene diisocyanate, or trimethylol propane-adduct of xylylene diisocyanate (commercially available from Mitsui Chemicals under the tradename Takenate® D-110N).

Aliphatic polyisocyanates may include a trimer of hexamethylene diisocyanate, a trimer of isophorone diisocyanate, a trimethylol propane-adduct of hexamethylene diisocyanate (available from Mitsui Chemicals) or a biuret of hexamethylene diisocyanate (commercially available from Bayer under the tradename Desmodur® N 100).

Derivatives of polyisocyanates may include oligomers or polymers of isocyanate monomers. As a non-limiting example, the polyisocyanate may preferably comprise an oligomer or polymer of diphenylmethane diisocyanate (MDI), such as Mondur® MR-Light.

The polyisocyanate may preferably be selected from the group consisting of: a polyisocyanurate of toluene diisocyanate; a trimethylol propane adduct of toluene diisocyanate; a trimethylol propane adduct of xylylene diisocyanate; 2,2′-methylenediphenyl diisocyanate; 4,4′-methylenediphenyl diisocyanate; 2,4′-methylenediphenyl diisocyanate; [diisocyanato(phenyl)methyl]benzene; toluene diisocyanate; tetramethylxylidene diisocyanate; naphthalene-1,5-diisocyanate; 1,4-phenylene diisocyanate; 1,3-diisocyanatobenzene; derivatives thereof (such as pre-polymers, oligomers, and/or polymers thereof); and combinations thereof.

The particle shell may also be reinforced using additional co-crosslinkers such as multifunctional amines and/or polyamines, such as diethylene triamine (DETA), polyethylene imine, polyvinyl amine, or mixtures thereof. Acrylates may also be used as additional co-crosslinkers, for example to reinforce the shell.

The polymeric material may be formed in a reaction, where the weight ratio of the biopolymer, preferably a polysaccharide, more preferably chitosan or a derivative thereof (which can include acid-treated chitosan) present in the reaction to the cross-linker present in the reaction is from about 1:10 to about 1:0.1, preferably from about 1:5 to about 5:1, preferably from about 1:4 to about 5:1, more preferably from about 1:1 to about 5:1, more preferably from about 3:1 to about 5:1 .. It is believed that selecting desirable ratios of the biopolymer to the cross-linking agent can provide desired ductility benefits, as well as improved biodegradability. It may be preferred that at least 21 wt % of the shell is comprised of moieties derived from chitosan, preferably from acid-treated chitosan. Chitosan as a percentage by weight of the shell may be from about 21% up to about 95% of the shell. The ratio of chitosan in the water phase as compared to the isocyanate in the oil phase may be, based on weight, from 21:79 to 90:10, or even from 1:2 to 9:1, or even from 1:1 to 7:1.

The shell may comprise chitosan at a level of 21 wt % or even greater, preferably from about 21 wt % to about 90 wt %, or even from 21 wt % to 85 wt %, or even 21 wt % to 75 wt %, or 21 wt % to 55 wt % of the total shell being chitosan. The chitosan of this paragraph may preferably be acid-treated chitosan.

The population of delivery particles may be made according to a process that comprises the following steps: (a) forming a water phase that includes chitosan as described herein, preferably where the water phase is at a pH of 6.5 or less, more preferably at a pH of from 3 to 6, and a temperature of at least 25° C.; (b) forming an oil phase that comprises at least one benefit agent, preferably fragrance material, and at least cross-linking agent, preferably at one polyisocyanate, and optionally a partitioning modifier; (c) forming an emulsion, preferably an oil-in-water emulsion, by mixing the water phase and the oil phase under high shear agitation, optionally adjusting the pH of the emulsion to be in a range of from pH 2 to pH 6, preferably pH 3 to pH 6; (d) curing the emulsion by heating, preferably to at least 40° C., for a time sufficient to form a shell at the interface of the oil droplets with the water phase, where the shell will comprise a polymeric material that is the reaction product of the chitosan and the cross-linking agent, and where the shell surrounds a core that comprises the benefit agent.

The population of delivery particles may be made according to a process that comprises the following steps: (a) forming a water phase by treating the chitosan with a mixture of a first acid and a second acid, the first acid comprising a strong acid, and the second acid comprising a weak acid, wherein the chitosan is treated at a pH of 6.5 or less, or even less than pH 6.5, or even at a pH of from 3 to 6, and a temperature of at least 25° C. for at least one hour; (b) forming an oil phase comprising dissolving together at least one benefit agent and at least one polyisocyanate, optionally with an added oil (e.g., partitioning modifier) and/or solvent; (c) forming an emulsion by mixing under high shear agitation the water phase and the oil phase into an excess of the water phase, thereby forming droplets of the oil phase dispersed in the water phase, and optionally adjusting the pH of the emulsion to be in a range from pH 2 to pH 6, preferably pH 3 to pH 6; (d) curing-the emulsion by heating to at least 40° C., for a time sufficient to form a shell at an interface of the droplets with the water phase, the shell comprising the reaction product of the polyisocyanate and the acid treated chitosan, and the shell surrounding the core comprising the droplets of the oil phase and benefit agent.

Chitosan may be added into water in a jacketed reactor and at pH from 2 or even from 3 to 6.5, adjusted using acid such as concentrated HCl. The chitosan of this mixture may be acid-treated by heating to elevated temperature, such as 85° C. in 60 minutes, and then may be held at this temperature from 1 minute to 1440 minutes or longer. The water phase then may be cooled to 25 ° C. Optionally, deacetylating may also be further facilitated or enhanced by enzymes to depolymerize or deacetylate the chitosan. An oil phase may be prepared by dissolving an isocyanate such as trimers of xylylene Diisocyanate (XDI) or polymers of methylene diphenyl isocyanate (MDI), in oil at 25° C. Diluents, for example isopropyl myristate, may be used to adjust the hydrophilicity of the oil phase. The oil phase may then be added into the water phase and milled at high speed to obtain a targeted size. The emulsion may then be cured in one or more heating steps, such as heating to 40° C. in 30 minutes and holding at 40° C. for 60 minutes. Times and temperatures are approximate. The temperature and time are selected to be sufficient to form and cure a shell at the interface of the droplets of the oil phase with the water continuous phase. For example, the emulsion may be heated to 85° C. in 60 minutes and then held at 85° C. for 360 minutes to cure the particles. The slurry may then be cooled to room temperature.

The shell may degrade at least 50% after 20 days (or less) when tested according to test method OECD 301B. The shell may degrade at least 60% of its mass after 60 days (or less) when tested according to test method OECD 301B. The shell may preferably degrade at least 60% of its mass after 60 days (or less) when tested according to test method OECD 301B. The shell may degrade from 30-100%, preferably 40-100%, 50-100%, 60-100%, or 60-95%, in 60 days, preferably 50 days, more preferably 40 days, more preferably 28 days, more preferably 14 days.

The delivery particles of the present disclosure include a core. The core comprises a fragrance material. The fragrance material comprises one or more perfume raw materials (PRMs). The core optionally comprises a partitioning modifier.

The core of a particle is surrounded by the shell. When the shell is ruptured, the fragrance material in the core is released. Additionally or alternatively, the fragrance material in the core may diffuse out of the particle, and/or it may be squeezed out. Suitable fragrance materials located in the core may include fragrance materials that provide benefits to a surface, such as a fabric or hair.

The core may comprise from about 5% to about 100%, by weight of the core, of a fragrance material. The core may comprise from about 45% to about 95%, preferably from about 50% to about 80%, more preferably from about 50% to about 70%, by weight of the core, of the fragrance material.

The fragrance material may be relatively hydrophobic. Such agents are compatible with the oil phases that are common in making the delivery particles of the present disclosure.

As described above, the cores of the delivery particles of the present disclosure comprise a fragrance, which may include one or more perfume raw materials. Fragrance is particularly suitable for encapsulation in the presently described delivery particles, as the fragrance-containing particles can provide freshness benefits across multiple touchpoints.

The term “perfume raw material” (or “PRM”) as used herein refers to compounds having a molecular weight of at least about 100 g/mol and which are useful in imparting an odor, fragrance, essence or scent, either alone or with other perfume raw materials. Typical PRMs comprise inter alia alcohols, ketones, aldehydes, esters, ethers, nitrites and alkenes, such as terpene. A listing of common PRMs can be found in various reference sources, for example, “Perfume and Flavor Chemicals”, Vols. I and II; Steffen Arctander Allured Pub. Co. (1994) and “Perfumes: Art, Science and Technology”, Miller, P. M. and Lamparsky, D., Blackie Academic and Professional (1994).

The PRMs may be characterized by their boiling points (B.P.) measured at the normal pressure (760 mm Hg), and their octanol/water partitioning coefficient (P), which may be described in terms of log P, determined according to the test method below. Based on these characteristics, the PRMs may be categorized as Quadrant I, Quadrant II, Quadrant III, or Quadrant IV perfumes, as described in more detail below.

The fragrance may comprise perfume raw materials that have a log P of from about 2.5 to about 4. It is understood that other perfume raw materials may also be present in the fragrance.

The perfume raw materials may comprise a perfume raw material selected from the group consisting of perfume raw materials having a boiling point (B.P.) lower than about 250° C. and a log P lower than about 3, perfume raw materials having a B.P. of greater than about 250° C. and a log P of greater than about 3, perfume raw materials having a B.P. of greater than about 250° C. and a log P lower than about 3, perfume raw materials having a B.P. lower than about 250° C. and a log P greater than about 3 and mixtures thereof. Perfume raw materials having a boiling point B.P. lower than about 250° C. and a log P lower than about 3 are known as Quadrant I perfume raw materials. Quadrant 1 perfume raw materials are preferably limited to less than 30% of the perfume composition. Perfume raw materials having a B.P. of greater than about 250° C. and a log P of greater than about 3 are known as Quadrant IV perfume raw materials, perfume raw materials having a B.P. of greater than about 250° C. and a log P lower than about 3 are known as Quadrant II perfume raw materials, perfume raw materials having a B.P. lower than about 250° C. and a log P greater than about 3 are known as a Quadrant III perfume raw materials. Suitable Quadrant I, II, III and IV perfume raw materials are disclosed in U.S. Pat. No. 6,869,923 B1.

As mentioned above, there are challenges associated with encapsulating certain PRMs in certain wall materials. It has surprisingly been found that effective delivery particles can be made by selecting certain PRMs to be encapsulated. For example, it has been found to be beneficial to select a certain minimum amount of ester-containing PRMs to be part of the fragrance material to be encapsulated. Additionally or alternatively, it has been found to be beneficial to select the components of the fragrance material so that the fragrance material is characterized by one or more S-ESTER values, described in more detail below.

The fragrance material may comprise at least 30%, by weight of the fragrance material, of ester-containing perfume raw materials (PRMs). Preferably, the fragrance material also contains at least 0.5%, by weight of the fragrance material, of aldehyde-containing PRMs. It is surprisingly been found that including a relatively high amount of ester-containing PRMs in the fragrance material allows for favorable encapsulation of the entire fragrance material. This is the case even when the fragrance material comprises aldehyde-containing PRMs, which are known to lead to encapsulation challenges when using amine-containing shell precursors.

The fragrance material may comprise at least about 30%, preferably about 35%, more preferably at least about 40%, even more preferably at least about 50%, by weight of the fragrance material, of ester-containing PRMs. The fragrance material may comprise from 30% to 99.5%, preferably from 35% to about 80%, more preferably from about 40% to about 70%, even more preferably from about 50% to about 60%, by weight of the fragrance material. It is believed that relatively higher amounts of ester-containing PRMs will lead to more effective encapsulation and/or performance.

Of note, certain PRMs include more than one functional group, such as an ester group and a hydroxyl group (see, e.g., triethyl citrate). For the purposes of the present disclosure, when determining the relative percentage of PRMs in a given fragrance material that include a given functional group, PRMs that have more than one type of functional group are counted in each functional groups category. Thus, the amount of triethyl citrate in a given fragrance would be counted towards both the percentage of ester-containing PRMs and the percentage of hydroxyl-containing PRMs. When such materials are present, the relative percentages of PRMs that have each functional group may add up to more than 100%, by weight of the fragrance material, due to such double (or even triple, etc.) counting.

Fragrance materials, including individual PRMs, can be described in terms of their atom level electrotopological state (E-State) indices. E-State values are molecular descriptors that can be found in literature and/or determined using commercially available software programs. The E-State values effectively look at each atom in a molecule and reflect the valence state electronegativity of the atom, which is affected by atoms connected to it. Electropological State indices are discussed more in Hall et al., Electrotopological State Indices for Atom Types: A Novel Combination of Electronic, Topological, and Valence State Information, J. Chem. Inf. Comput. Sci. 1995, 35, 1039-1045.

The S-ESTER value is the sum of the atom level electrotopological state (E-State) values for all carbonyl carbons and oxygens in ester groups in the molecule. To determine E-State and S-ESTER values for the purposes described in the present disclosure, E-State values are computed using software program winMolconn version 1.2.2.3 (available from Hall Associates Consulting of Quincy, MA), used according to the manufacturer's instructions. The method to determine the S-ESTER value is described in more detail in the Test Methods section below.

Relatively high S-ESTER values for a particular compound may indicate, for example, more than one ester group. Relatively high S-ESTER values for a mixture of compounds, such as a fragrance material that comprises more than one type of perfume raw material, can indicate a relatively high proportion of ester-containing compounds, including compounds that comprise more than one ester group.

The fragrance material that is encapsulated in the delivery particles of the present disclosure may be characterized according to the S-ESTER value of the entire fragrance material (e.g., the mixture of PRMs), and/or by the relative amount of PRMs that are characterized by a certain S-ESTER value. For example, the fragrance material may be characterized by one or more of the following: (a) as having an S-ESTER value of at least about 5, wherein the S-ESTER value of the fragrance material is calculated as the weight average S-ESTER values of the perfume raw materials; and/or (b) as comprising at least about 30%, by weight of the fragrance materials, of perfume raw materials having S-ESTER values of 13 or greater.

The fragrance material may be characterized as having an S-ESTER value of at least about 5, preferably at least about 5.5, preferably at least about 6. Additionally, the fragrance material may be preferably characterized by an S-ESTER value of no greater than about 20, preferably no greater than about 13, more preferably no greater than about 10. Greater S-ESTER values are associated with a relatively greater amount of esters, which may be associated with relatively lower volatility and lower olfactory impact.

The fragrance material may comprise at least about 30%, preferably at least about 40%, more preferably at least about 50%, even more preferably at least about 55%, by weight of the fragrance materials, of perfume raw materials having S-ESTER values of about 13 or greater, preferably PRMs having S-ESTER values of from about 13 to about 80, more preferably from about 13 to about 35, even more preferably from about 13 to about 20. For clarity, it is understood that PRMs that do not include an ester group are assigned an S-ESTER value of zero.

Suitable ester-containing PRMs for use in the fragrance materials of the present disclosure are provided in the following table. The fragrance material of the present disclosure may comprise any of the PRMs listed in the following table, or combinations thereof. The table also indicates what type of functional groups are present, as well as the S-ESTER value assigned to each PRM.

| | | S-ESTER |
|---|---|---|
| Perfume Raw Material | Functionality | value |
| 4-TERTIARY BUTYL | ESTER | 15.8 |
| CYCLOHEXYL ACETATE | | |
| ALLYL AMYL GLYCOLATE | ESTER + ETHER | 15.2 |
| ALLYL CAPROATE | ESTER | 15.5 |
| ALLYL CYCLOHEXANE | ESTER | 16.0 |
| PROPIONATE | | |
| ALLYL HEPTOATE | ESTER | 15.6 |
| AMYL- ACETATE (isomer | ESTER | 14.7 |
| blends) | | |
| AMYL SALICYLATE | ESTER + HYDROXYL | 16.0 |
| CIS 3 HEXENYL ACETATE | ESTER | 14.7 |
| CIS-3-Hexenyl Salicylate | ESTER + HYDROXYL | 16.0 |
| Cyclabute | ESTER | 17.3 |
| Cyclo Galbanate | ESTER + ETHER | 15.6 |
| CYCLOBUTANATE | ESTER | 17.1 |
| DIHYDRO TERPINYL | ESTER | 16.2 |
| ACETATE | | |
| DIMETHYL BENZYL | ESTER | 15.8 |
| CARBINYL ACETATE | | |
| DIMETHYL BENZYL | ESTER | 16.8 |
| CARBINYL BUTYRATE | | |
| EXALTOLIDE TOTAL | ESTER | 16.6 |
| ETHYL 2 METHYL | ESTER | 15.7 |
| PENTANOATE | | |
| ETHYL ACETATE | ESTER | 14.0 |
| ETHYL BUTYRATE | ESTER | 15.0 |
| ETHYLENE BRASSYLATE | ESTER | 32.3 |
| ETHYL ISOVALERATE | ESTER | 15.3 |
| ETHYL LAITONE | ESTER | 16.5 |
| ETHYL METHYL PHENYL | ESTER + ETHER | 16.1 |
| GLYCIDATE | | |
| ETHYL-2-METHYL | ESTER | 15.4 |
| BUTYRATE | | |
| FLOR ACETATE | ESTER | 16.2 |
| FRUCTALATE | ESTER | 32.6 |
| FRUTENE | ESTER | 16.8 |
| GAMMA DECALACTONE | ESTER | 15.8 |
| GERANYL ACETATE | ESTER | 15.1 |
| Habanolide | ESTER | 16.5 |
| HELVETOLIDE | ESTER + ETHER | 16.3 |
| HEXYL ACETATE | ESTER | 14.8 |
| ISO BORNYL ACETATE | ESTER | 16.3 |
| ISO NONYL ACETATE | ESTER | 15.2 |
| ISOAMYL BUTYRATE | ESTER | 15.7 |
| Jasmal | ESTER + KETONE | 16.1 |
| METHYL ANTHRANILATE | ESTER | 15.0 |
| METHYL BENZOATE | ESTER | 15.0 |
| METHYL DIHYDRO | ESTER + KETONE | 15.7 |
| JASMONATE | | |
| METHYL DIOXOLAN | ESTER + ETHER | 15.5 |
| METHYL LAITONE | ESTER | 16.4 |
| METHYL PHENYL CARBINYL | ESTER | 15.4 |
| ACETATE | | |
| Musk RI | ESTER + ETHER | 16.6 |
| Octahydro Coumarin | ESTER | 16.2 |
| PHENOXY ETHYL ISO | ESTER + ETHER | 15.8 |
| BUTYRATE | | |
| PHENYL ETHYL TIGLATE | ESTER | 16.1 |
| PRENYL ACETATE | ESTER | 14.6 |
| Romandolide | ESTER | 31.8 |
| SCLAREOLATE | ESTER + ETHER | 16.1 |
| Terpinyl Acetate | ESTER | 16.1 |
| Thesaron | ESTER | 17.0 |
| Triethyl citrate | ESTER + HYDROXYL | 45.7 |
| UNDECALACTONE | ESTER | 15.9 |
| VERDOX | ESTER | 16.2 |
| Violiff | ESTER | 30.0 |
| Zenolide | ESTER | 32.2 |

Even though it is believed that relatively higher amounts of ester-containing PRMs will lead to more effective encapsulation and/or performance, it may be desirable to include PRMs having non-ester moieties in the interest of forming a well-rounded fragrance from an olfactory point of view.

For example, the fragrance material may comprise aldehyde-containing PRMs. The fragrance material may comprise at least about 0.5%, preferably at least about 1%, more preferably at least about 5%, more preferably at least about 10%, by weight of the fragrance material, of aldehyde-containing PRMs. It may be desirable to limit the amount of aldehyde-containing PRMs due to the known issues related to encapsulation. For example, the fragrance material may comprise from 0.5% to about 30%, preferably from about 1% to about 25%, more preferably from about 5% to about 20%, by weight of the fragrance material, of aldehyde-containing PRMs. Aldehyde-containing perfume raw materials may include: methyl nonyl acetaldehyde: benzaldehyde; floralozone; isocyclocitral; triplal (ligustral); precyclemone B; lilial; decyl aldehyde; undecylenic aldehyde; cyclamen homoaldehyde; cyclamen aldehyde; dupical; oncidal; adoxal; melonal; calypsone; anisic aldehyde; heliotropin; cuminic aldehyde; scentenal; 3,6-dimethylcyclohex-3-ene-1-carbaldehyde; satinaldehyde; canthoxal; vanillin; ethyl vanillin; cinnamic aldehyde; cis-4-decenal; trans-4-decenal; cis-7-decenal; undecylenic aldehyde; trans-2-hexenal; trans-2-octenal; 2-undecenal; 2,4-dodecadeienal; cis-4-heptenal; Florydral; butyl cinnamaldehyde; limonelal; amyl cinnamaldehyde; hexyl cinnamaldehyde; citronellal; citral; cis-3-hexen-1-al; or mixtures thereof, as these materials are particularly desired by formulators and/or consumers.

The fragrance material may comprise hydroxyl-containing PRMs. However, to promote effective shell formation, the relative amount of such PRMs may be limited. The fragrance material may comprise no more than 30%, by weight of the fragrance materials, of hydroxyl-containing PRMs, preferably from 1% to 30% of hydroxyl-containing PRMs. Hydroxyl-containing perfume raw materials may include: geraniol, nonadienol, cinnamic alcohol, linalool, flor acetate, derivatives thereof, or mixtures thereof, as these materials are particularly desired by formulators and/or consumers.

The fragrance material may comprise ketone-containing PRMs. Ketone-containing perfume raw materials may include: nerolione; 4-(4-methoxyphenyl)butan-2-one; 1-naphthalen-2-ylethanone; nectaryl; trimofix O; fleuramone; delta-damascone; beta-damascone; alpha-damascone; methyl ionone; 2-hexylcyclopent-2-en-1-one; galbascone; or mixtures thereof, as these materials are particularly desired by formulators and/or consumers.

A preferred fragrance material suitable to be encapsulated in the delivery particles of the present disclosure may include the following categories of PRMs in the following amounts: from about 45% to about 55% of ester-containing PRMs; from about 10% to about 25% of aldehyde-containing PRMs; from about 5% to about 20% hydroxyl-containing PRMs; and optionally from about 1% to about 40% of additional PRMs, which may include PRMs that are ketone-containing PRMs.

The core of the delivery particles of the present disclosure may comprise a partitioning modifier, which may facilitate more robust shell formation. The partitioning modifier may be combined with the core's perfume oil material prior to incorporation of the wall-forming monomers. The partitioning modifier may be present in the core at a level of from 0% to 95%, preferably from about 5% to about 55%, preferably from about 10% to about 50%, more preferably from about 25% to about 50%, by weight of the core.

The partitioning modifier may comprise a material selected from the group consisting of vegetable oil, modified vegetable oil, mono-, di-, and tri-esters of C₄-C₂₄ fatty acids, isopropyl myristate, dodecanophenone, lauryl laurate, methyl behenate, methyl laurate, methyl palmitate, methyl stearate, and mixtures thereof. The partitioning modifier may preferably comprise or even consist of isopropyl myristate. The modified vegetable oil may be esterified and/or brominated. The modified vegetable oil may preferably comprise castor oil and/or soy bean oil. US Patent Application Publication 20110268802, incorporated herein by reference, describes other partitioning modifiers that may be useful in the presently described delivery particles.

Optionally, the core may comprise additional benefit agents, which may be selected from the group consisting of silicone oils, waxes, hydrocarbons, higher fatty acids, essential oils, lubricants, lipids, skin coolants, vitamins, sunscreens, antioxidants, glycerine, catalysts, bleach particles, silicon dioxide particles, malodor reducing agents, odor-controlling materials, antistatic agents, softening agents, insect and moth repelling agents, colorants, antioxidants, chelants, bodying agents, drape and form control agents, smoothness agents, wrinkle control agents, sanitization agents, disinfecting agents, germ control agents, mold control agents, mildew control agents, antiviral agents, drying agents, stain resistance agents, soil release agents, fabric refreshing agents and freshness extending agents, chlorine bleach odor control agents, dye fixatives, dye transfer inhibitors, color maintenance agents, optical brighteners, color restoration/rejuvenation agents, anti-fading agents, whiteness enhancers, anti-abrasion agents, wear resistance agents, fabric integrity agents, anti-wear agents, anti-pilling agents, defoamers, anti-foaming agents, UV protection agents, sun fade inhibitors, anti-allergenic agents, enzymes, water proofing agents, fabric comfort agents, shrinkage resistance agents, stretch resistance agents, stretch recovery agents, skin care agents, synthetic or natural actives, antibacterial actives, antiperspirant actives, cationic polymers, dyes, and mixtures thereof.

Where the benefit agent is not itself sufficient to serve as the oil phase or solvent, particularly during the process of forming the shell of the delivery particles for the wall forming materials, the oil phase can comprise a suitable carrier and/or solvent. In this sense, the oil is optional, as the benefit agent itself can at times be the oil. These carriers or solvents are generally an oil, preferably have a boiling point greater than about 80° C. and low volatility and are non-flammable. Though not limited thereto, they preferably comprise one or more esters, preferably with chain lengths of up to 18 carbon atoms or even up to 42 carbon atoms and/or triglycerides such as the esters of C6 to C12 fatty acids and glycerol.

Optionally, the water phase may include an emulsifier. Non-limiting examples of emulsifiers include anionic surfactants (such as alkyl sulfates, alkyl ether sulfates, and/or alkyl benzenesulfonates), nonionic surfactants (such as alkoxylated alcohols, preferably comprising ethoxy groups), polyvinyl alcohol, and/or polyvinyl pyrrolidone. It may be that solubilized chitosan can provide emulsifying benefits in the present applications.

Emulsifier, if employed, is typically from about 0.1 to 40% by weight, preferably 0.2 to about 15% by weight, more typically 0.5 to 10% be weight, based on total weight of the aqueous phase.

The population of delivery particles may be provided as a slurry, preferably an aqueous slurry. The slurry can include one or more processing aids, which may include water, aggregate inhibiting materials such as divalent salts, or particle suspending polymers such as xanthan gum, guar gum, cellulose (preferably microfibrillated cellulose) and/or carboxy methyl cellulose. When the delivery particles are characterized by a cationic nature (for example, when the shell is derived, at least in part, from chitosan), a non-anionic structurant, preferably a nonionic structurant, may be preferred, for example, to avoid detrimental charge interactions that may lead to undesirable aggregation.

The slurry can include one or more carriers selected from the group consisting of polar solvents, including but not limited to, water, ethylene glycol, propylene glycol, polyethylene glycol, glycerol; nonpolar solvents, including but not limited to, mineral oil, perfume raw materials, silicone oils, hydrocarbon paraffin oils; and mixtures thereof. Aqueous slurries may be preferred. The slurry may comprise non-encapsulated (of “free”) perfume raw materials that are different in identity and/or amount from those that are encapsulated in the cores of the delivery particles.

The slurry may include a deposition aid that may comprise a polymer selected from the group comprising: polysaccharides, in one aspect, cationically modified starch and/or cationically modified guar; polysiloxanes; poly diallyl dimethyl ammonium halides; copolymers of poly diallyl dimethyl ammonium chloride and polyvinyl pyrrolidone; a composition comprising polyethylene glycol and polyvinyl pyrrolidone; acrylamides; imidazoles; imidazolinium halides; polyvinyl amine; copolymers of poly vinyl amine and N-vinyl formamide; polyvinyl formamide, polyvinyl alcohol; polyvinyl alcohol crosslinked with boric acid; polyacrylic acid; polyglycerol ether silicone cross-polymers; polyacrylic acids, polyacrylates, copolymers of polyvinylamine and polvyinylalcohol oligomers of amines, in one aspect a diethylenetriamine, ethylene diamine, bis(3-aminopropyl)piperazine, N,N-Bis-(3-aminopropyl)methylamine, tris(2-aminoethyl)amine and mixtures thereof; polyethyleneimine, a derivatized polyethyleneimine, in one aspect an ethoxylated polyethyleneimine; a polymeric compound comprising, at least two moieties selected from the moieties consisting of a carboxylic acid moiety, an amine moiety, a hydroxyl moiety, and a nitrile moiety on a backbone of polybutadiene, polyisoprene, polybutadiene/styrene, polybutadiene/acrylonitrile, carboxyl-terminated polybutadiene/acrylonitrile or combinations thereof; pre-formed coacervates of anionic surfactants combined with cationic polymers; polyamines and mixtures thereof.

At least one population of delivery particles may be contained in an agglomerate and then combined with a distinct population of delivery particles and at least one adjunct material. Said agglomerate may comprise materials selected from the group consisting of silicas, citric acid, sodium carbonate, sodium sulfate, sodium chloride, and binders such as sodium silicates, modified celluloses, polyethylene glycols, polyacrylates, polyacrylic acids, zeolites and mixtures thereof.

Suitable equipment for use in the processes disclosed herein may include continuous stirred tank reactors, homogenizers, turbine agitators, recirculating pumps, paddle mixers, plough shear mixers, ribbon blenders, vertical axis granulators and drum mixers, both in batch and, where available, in continuous process configurations, spray dryers, and extruders. Such equipment can be obtained from Lodige GmbH (Paderborn, Germany), Littleford Day, Inc. (Florence, Ky., U.S.A.), Forberg AS (Larvik, Norway), Glatt Ingenieurtechnik GmbH (Weimar, Germany), Niro

(Socborg, Denmark), Hosokawa Bepex Corp. (Minneapolis, Minn., U.S.A.), Arde Barinco (New Jersey, U.S.A.).

### Adjunct Ingredient

The treatment compositions of the present disclosure may comprise one or more adjunct materials in addition to the delivery particles. The adjunct material may provide a benefit in the intended end-use of a composition, or it may be a processing and/or stability aid.

Suitable adjunct materials may include: surfactants, conditioning actives, deposition aids, rheology modifiers or structurants, bleach systems, stabilizers, builders, chelating agents, dye transfer inhibiting agents, dispersants, enzymes, and enzyme stabilizers, catalytic metal complexes, polymeric dispersing agents, clay and soil removal/anti-redeposition agents, brighteners, suds suppressors, silicones, hucing agents, aesthetic dyes, additional perfumes and perfume delivery systems, structure elasticizing agents, carriers, hydrotropes, processing aids, anti-agglomeration agents, coatings, formaldehyde scavengers, and/or pigments. Preferably, the adjunct materials comprise additional fabric conditioning agents, dyes, pH control agents, solvents, rheology modifiers, structurants, cationic polymers, surfactants, perfume, additional perfume delivery systems, chelants, antioxidants, preservatives, or mixtures thereof.

Depending on the intended form, formulation, and/or end-use, compositions of the present disclosure might not contain one or more of the following adjuncts materials: bleach activators, surfactants, builders, chelating agents, dye transfer inhibiting agents, dispersants, enzymes, and enzyme stabilizers, catalytic metal complexes, polymeric dispersing agents, clay and soil removal/anti-redeposition agents, brighteners, suds suppressors, dyes, additional perfumes and perfume delivery systems, structure clasticizing agents, fabric softeners, carriers, hydrotropes, processing aids, structurants, anti-agglomeration agents, coatings, formaldehyde scavengers, and/or pigments.

The precise nature of these additional components, and levels of incorporation thereof, will depend on the physical form of the composition and the nature of the operation for which it is to be used. However, when one or more adjuncts are present, such one or more adjuncts may be present as detailed below. The following is a non-limiting list of suitable additional adjuncts.

### A. Surfactants

The compositions of the present disclosure may comprise surfactant. Surfactants may be useful for providing, for example, cleaning benefits. The compositions may comprise a surfactant system, which may contain one or more surfactants.

The compositions of the present disclosure may include from about 0.1% to about 70%, or from about 2% to about 60%, or from about 5% to about 50%, by weight of the composition, of a surfactant system. Liquid compositions may include from about 5% to about 40%, by weight of the composition, of a surfactant system. Compact formulations, including compact liquids, gels, and/or compositions suitable for a unit dose form, may include from about 25% to about 70%, or from about 30% to about 50%, by weight of the composition, of a surfactant system.

The surfactant system may include anionic surfactant, nonionic surfactant, zwitterionic surfactant, cationic surfactant, amphoteric surfactant, or combinations thereof. The surfactant system may include linear alkyl benzene sulfonate, alkyl ethoxylated sulfate, alkyl sulfate, nonionic surfactant such as ethoxylated alcohol, amine oxide, or mixtures thereof. The surfactants may be, at least in part, derived from natural sources, such as natural feedstock alcohols.

Suitable anionic surfactants may include any conventional anionic surfactant. This may include a sulfate detersive surfactant, for e.g., alkoxylated and/or non-alkoxylated alkyl sulfate materials, and/or sulfonic detersive surfactants, e.g., alkyl benzene sulfonates. The anionic surfactants may be linear, branched, or combinations thereof. Preferred surfactants include linear alkyl benzene sulfonate (LAS), alkyl ethoxylated sulfate (AES), alkyl sulfates (AS), or mixtures thereof. Other suitable anionic surfactants include branched modified alkyl benzene sulfonates (MLAS), methyl ester sulfonates (MES), sodium lauryl sulfate (SLS), sodium lauryl ether sulfate (SLES), and/or alkyl ethoxylated carboxylates (AEC). The anionic surfactants may be present in acid form, salt form, or mixtures thereof. The anionic surfactants may be neutralized, in part or in whole, for example, by an alkali metal (e.g., sodium) or an amine (e.g., monoethanolamine). Due to the presence of cationic ester quat material, it may be desirable to limit the amount of anionic surfactant so as to avoid undesirable interactions of the materials; for example, the compositions may comprise less than 5%, preferably less than 3%, more preferably less than 1%, even more preferably less than 0.1%, by weight of the composition, of anionic surfactant.

The surfactant system may include nonionic surfactant. Suitable nonionic surfactants include alkoxylated fatty alcohols, such as ethoxylated fatty alcohols. Other suitable nonionic surfactants include alkoxylated alkyl phenols, alkyl phenol condensates, mid-chain branched alcohols, mid-chain branhed alkyl alkoxylates, alkylpolysaccharides (e.g., alkylpolyglycosides), polyhydroxy fatty acid amides, ether capped poly(oxyalkylated) alcohol surfactants, and mixtures thereof. The alkoxylate units may be ethyleneoxy units, propyleneoxy units, or mixtures thereof. The nonionic surfactants may be linear, branched (e.g., mid-chain branched), or a combination thereof. Specific nonionic surfactants may include alcohols having an average of from about 12 to about 16 carbons, and an average of from about 3 to about 9 ethoxy groups, such as C12-C14 EO7 nonionic surfactant.

Suitable zwitterionic surfactants may include any conventional zwitterionic surfactant, such as betaines, including alkyl dimethyl betaine and cocodimethyl amidopropyl betaine, Co to C₁₈ (for example from C₁₂ to C₁₈) amine oxides (e.g., C₁₂₋₁₄ dimethyl amine oxide), and/or sulfo and hydroxy betaines, such as N-alkyl-N,N-dimethylammino-1-propane sulfonate where the alkyl group can be C₈ to C₁₈, or from C₁₀ to C₁₄. The zwitterionic surfactant may include amine oxide.

Depending on the formulation and/or the intended end-use, the composition may be substantially free of certain surfactants. For example, liquid fabric enhancer compositions, such as fabric softeners, may be substantially free of anionic surfactant, as such surfactants may negatively interact with cationic ingredients.

### B. Conditioning Active

The compositions of the present disclosure may include a conditioning active. Compositions that contain conditioning actives may provide softness, anti-wrinkle, anti-static, conditioning, anti-stretch, color, and/or appearance benefits.

Conditioning actives may be present at a level of from about 1% to about 99%, by weight of the composition. The composition may include from about 1%, or from about 2%, or from about 3%, to about 99%, or to about 75%, or to about 50%, or to about 40%, or to about 35%, or to about 30%, or to about 25%, or to about 20%, or to about 15%, or to about 10%, by weight of the composition, of conditioning active. The composition may include from about 5% to about 30%, by weight of the composition, of conditioning active.

Conditioning actives suitable for compositions of the present disclosure may include quaternary ammonium ester compounds, silicones, non-ester quaternary ammonium compounds, amines, fatty esters, sucrose esters, silicones, dispersible polyolefins, polysaccharides, fatty acids, softening or conditioning oils, polymer latexes, or combinations thereof. Preferably the treatment composition is a fabric care composition where the one or more adjunct ingredients comprises quaternary ammonium ester material; such materials are particularly useful in fabric enhancing/conditioning/softening compositions.

The composition may include a quaternary ammonium ester compound, a silicone, or combinations thereof, preferably a combination. The combined total amount of quaternary ammonium ester compound and silicone may be from about 5% to about 70%, or from about 6% to about 50%, or from about 7% to about 40%, or from about 10% to about 30%, or from about 15% to about 25%, by weight of the composition. The composition may include a quaternary ammonium ester compound and silicone in a weight ratio of from about 1:10 to about 10:1, or from about 1:5 to about 5:1, or from about 1:3 to about 1:3, or from about 1:2 to about 2:1, or about 1:1.5 to about 1.5:1, or about 1:1.

The composition may contain mixtures of different types of conditioning actives. The compositions of the present disclosure may contain a certain conditioning active but be substantially free of others. For example, the composition may be free of quaternary ammonium ester compounds, silicones, or both. The composition may comprise quaternary ammonium ester compounds but be substantially free of silicone. The composition may comprise silicone but be substantially free of quaternary ammonium ester compounds.

### C. Deposition Aid

The compositions of the present disclosure may comprise a deposition aid. As described above, due to the synergistic benefits that flow from the ester quat material and the delivery particles of the present disclosure, relatively less (or even none) of a deposition aid may be require to provide comparable or even improved performance; alternatively, a deposition aid may be used in compositions of the present disclosure to boost performance even more.

Deposition aids can facilitate deposition of delivery particles, conditioning actives, perfumes, or combinations thereof, improving the performance benefits of the compositions and/or allowing for more efficient formulation of such benefit agents. The composition may comprise, by weight of the composition, from 0.0001% to 3%, preferably from 0.0005% to 2%, more preferably from 0.001% to 1%, or from about 0.01% to about 0.5%, or from about 0.05% to about 0.3%, of a deposition aid. The deposition aid may be a cationic or amphoteric polymer, preferably a cationic polymer.

Cationic polymers in general and their methods of manufacture are known in the literature. Suitable cationic polymers may include quaternary ammonium polymers known the “Polyquaternium” polymers, as designated by the International Nomenclature for Cosmetic Ingredients, such as Polyquaternium-6 (poly(diallyldimethylammonium chloride), Polyquaternium-7 (copolymer of acrylamide and diallyldimethylammonium chloride), Polyquaternium-10 (quaternized hydroxyethyl cellulose), Polyquaternium-22 (copolymer of acrylic acid and diallyldimethylammonium chloride), and the like.

The deposition aid may be selected from the group consisting of polyvinylformamide, partially hydroxylated polyvinylformamide, polyvinylamine, polyethylene imine, ethoxylated polyethylene imine, polyvinylalcohol, polyacrylates, and combinations thereof. The cationic polymer may comprise a cationic acrylate.

Deposition aids can be added concomitantly with delivery particles (at the same time with, e.g., encapsulated benefit agents) or directly/independently in the consumer product composition. The weight-average molecular weight of the polymer may be from 500 to 5000000 or from 1000 to 2000000 or from 2500 to 1500000 Dalton, as determined by size exclusion chromatography relative to polyethyleneoxide standards using Refractive Index (RI) detection. The weight-average molecular weight of the cationic polymer may be from 5000 to 37500 Dalton.

### D. Rheology Modifier/Structurant

The compositions of the present disclosure may contain a rheology modifier and/or a structurant. Rheology modifiers may be used to “thicken” or “thin” liquid compositions to a desired viscosity. Structurants may be used to facilitate phase stability and/or to suspend or inhibit aggregation of particles in liquid composition, such as the delivery particles as described herein.

Suitable rheology modifiers and/or structurants may include non-polymeric crystalline hydroxyl functional structurants (including those based on hydrogenated castor oil), polymeric structuring agents, cellulosic fibers (for example, microfibrillated cellulose, which may be derived from a bacterial, fungal, or plant origin, including from wood), di-amido gellants, or combinations thereof.

Polymeric structuring agents may be naturally derived or synthetic in origin. Naturally derived polymeric structurants may comprise hydroxyethyl cellulose, hydrophobically modified hydroxyethyl cellulose, carboxymethyl cellulose, polysaccharide derivatives and mixtures thereof. Polysaccharide derivatives may comprise pectine, alginate, arabinogalactan (gum Arabic), carrageenan, gellan gum, xanthan gum, guar gum and mixtures thereof. Synthetic polymeric structurants may comprise polycarboxylates, polyacrylates, hydrophobically modified ethoxylated urethanes, hydrophobically modified non-ionic polyols and mixtures thereof. Polycarboxylate polymers may comprise a polyacrylate, polymethacrylate or mixtures thereof. Polyacrylates may comprise a copolymer of unsaturated mono- or di-carbonic acid and C₁-C₃₀ alkyl ester of the (meth)acrylic acid. Such copolymers are available from Noveon inc under the tradename Carbopol Aqua 30. Cross-linked polymers, such as cross-linked polyacrylate and/or polymers and/or co-polymers, such as those that further include nonionic monomers such as acrylamide or methacrylamide monomers, may be useful as structurants. Another suitable structurant is sold under the tradename Rheovis CDE, available from BASF.

### E. Other Adjuncts

The treatment compositions of the present disclosure may contain other adjuncts that are suitable for inclusion in the product and/or for final usage. For example, the treatment compositions may comprise neat perfume, perfume delivery technologies (such as pro-perfumes and/or encapsulates having non-cross-linked-biopolymer wall materials), cationic surfactants, cationic polymers, solvents, suds supressors, or combinations thereof.

### Method of Making a Treatment Composition

The present disclosure further relates to methods for making a treatment composition, such as those treatment compositions and/or consumer product compositions described herein.

The method may comprise the steps of: providing a base composition, wherein the base composition comprises the treatment adjunct, and combining the population of delivery particles with the base composition. The population of delivery particles may preferably be provided as an aqueous slurry. The base composition is in the form of a liquid composition.

The delivery particles may be combined with the one or more adjunct ingredients when the delivery particles are in one or more forms, including a slurry form, neat particle form, and/or spray dried particle form, preferably slurry form. The delivery particles may be combined with such adjuncts by methods that include mixing and/or spraying.

The treatment compositions of the present disclosure can be formulated into any suitable form and prepared by any process chosen by the formulator. The one or more adjunct ingredients and the delivery particles may be combined in a batch process, in a circulation loop process, and/or by an in-line mixing process. Suitable equipment for use in the processes disclosed herein may include continuous stirred tank reactors, homogenizers, turbine agitators, recirculating pumps, paddle mixers, high shear mixers, static mixers, plough shear mixers, ribbon blenders, vertical axis granulators and drum mixers, both in batch and, where available, in continuous process configurations, spray dryers, and extruders.

The treatment composition may be placed into a container to form a consumer product, as described herein. The container may be a bottle, preferably a plastic bottle. The treatment composition may be placed into an aerosol or other spray container according to known methods.

### Method of Treatment

The present disclosure also relates to a method of treating a surface, preferably a fabric. In general, the method includes the step of contacting a surface, preferably a fabric, with a treatment composition according to the present disclosure, where the treatment composition includes a population of delivery particles as described herein.

Additionally or alternatively, the method may include the step of contacting a surface, preferably a fabric, with a population of delivery particles as described herein. The population of delivery particles may be contained in a treatment composition according to the present disclosure, preferably a fabric care composition.

The method may include the step of contacting a fabric, such as a garment, with a treatment composition. The treatment composition comprises a population of delivery particles. The contacting step results in one or more of the delivery particles being deposited on a surface of the fabric. The delivery particles comprise a core and a shell surrounding the core, where the core comprises a benefit agent, preferably a fragrance material that comprises one or more perfume raw materials. The shell comprises a polymeric material that is, for example, the reaction product of chitosan of a particular molecular weight and a cross-linking agent. Suitable treatment compositions and delivery particles are described in more detail above.

The contacting step may occur during a manual laundry process, for example in a wash basin as fabrics are treated by hand, or an automatic laundry process, for example in an automatic washing machine. The contacting step may occur during the wash cycle of an automatic washing machine; in such cases, the treatment composition may be a laundry detergent or a laundry additive. The contacting step may preferably occur during the rinse cycle of an automatic washing machine; in such cases, the treatment composition may be a fabric enhancer, preferably a liquid fabric enhancer. The contacting step may even occur during a drying step of a laundry process, for example in an automatic dryer machine; in such cases, the treatment composition may be in the form of a non-woven dryer sheet or a dryer bar. The contacting step may occur as a result of the treatment composition being directly applied to the fabric, for example in a pretreatment operation or in a “refreshing” step (e.g., for a fabric that has been used or worn since the last wash); in such cases, the treatment composition may be in the form of a liquid, a stick, or a spray, preferably a spray. Contacting the target fabrics relatively late in a laundering process, e.g., during a rinse cycle, improves the likelihood or efficiency of deposition onto the fabrics as they are less likely to be washed down the drain.

The contacting step may occur in the presence of water. The treatment composition may be diluted with water to form a treatment liquor. The treatment composition may be diluted from about 100-fold to about 1500-fold, preferably from 300-fold to about 1000-fold.

Liquors that comprise the disclosed compositions may have a pH of from about 3 to about 11.5. When diluted, such compositions are typically employed at concentrations of from about 500 ppm to about 15,000 ppm in solution. When the wash solvent is water, the water temperature typically ranges from about 5° C. to about 90° C. and, the water to fabric ratio may be typically from about 1:1 to about 30:1.

The dilution may occur in the drum of an automatic washing machine. The treatment composition may be placed into a dispensing drawer of an automatic washing machine. The treatment composition may be dispensed from the dispensing drawer to the drum during a treatment process.

As alluded to above, the method may further comprise a step of drying the fabric that has the one or more delivery particles on the surface of the fabric. The drying step may comprise a passive drying process, such as on a clothesline or drying rack. The drying step may comprise an automatic drying process, such as in an automatic dryer machine.

### COMBINATIONS

Specifically contemplated combinations of the disclosure are herein described in the following lettered paragraphs. These combinations are intended to be illustrative in nature and are not intended to be limiting.
A. A treatment composition comprising: a treatment adjunct, and a population of delivery particles, wherein the delivery particles comprise a core and a shell surrounding the core, wherein the shell comprises a polymeric material, wherein the polymeric material comprises a reaction product of a biopolymer and a cross-linking agent, wherein the biopolymer comprises primary amine groups, wherein the core comprises a fragrance material, wherein the fragrance material comprises: (a) at least about 30%, by weight of the fragrance material, of ester-containing perfume raw materials (“PRMs”), and (b) at least about 0.5wt %, by weight of the fragrance material, aldehyde-containing PRMs.
B. A treatment composition comprising: a treatment adjunct, and a population of delivery particles, wherein the delivery particles comprise a core and a shell surrounding the core, wherein the shell comprises a polymeric material, wherein the polymeric material comprises reaction product of a biopolymer and a cross-linking agent, wherein the biopolymer comprises primary amine groups, wherein the core comprises a fragrance material, wherein the fragrance material comprises one or more perfume raw materials (“PRMs”), and wherein the fragrance material is characterized by one or more of the following: (a) as having an S-ESTER value of at least about 5, wherein the S-ESTER value of the fragrance material is calculated as the weight average S-ESTER values of the perfume raw materials; and/or (b) as comprising at least about 30%, by weight of the fragrance materials, of perfume raw materials having S-ESTER values of 13 or greater.
C. The treatment composition according to any of paragraphs A or B, wherein the fragrance material comprises at least about 35%, preferably at least about 40%, preferably at least about 50%, by weight of the fragrance material, of ester-containing PRMs.
D. The treatment composition according to any of paragraphs A-C, wherein the fragrance material is characterized as having an S-ESTER value of at least about 5.5, preferably at least about 6, preferably wherein the S-ESTER value is no greater than about 20, preferably no greater than about 13, more preferably no greater than about 10.
E. The treatment composition according to any of paragraphs A-D, wherein the fragrance material comprises at least about 40%, preferably at least about 50%, more preferably at least about 55%, by weight of the fragrance materials, of perfume raw materials having S-ESTER values of about 13 or greater, preferably from about 13 to about 80, more preferably from about 13 to about 35, even more preferably from about 13 to about 20.
F. The treatment composition according to any of paragraphs A-E, wherein the ester-containing perfume raw materials comprise one or more materials selected from the group consisting of: 4-tertiary butyl cyclohexyl acetate; allyl amyl glycolate; allyl caproate; allyl cyclohexane propionate; allyl heptoate; amyl-acetate (isomer blends); Cis-3-hexenyl acetate; cis-3-hexenyl salicylate; cyclobute; cyclo galbanate; cyclobutanate; dihydro terpinyl acetate; dimethyl benzyl carbinyl acetate; dimethyl benzyl carbinyl butyrate; exaltolide total; ethyl 2 methyl pentanoate; ethyl acetate; ethyl butyrate; ethylene brassylate; ethyl isovalerate; ethyl laitone; ethyl methyl phenyl glycidate; ethyl methyl phenyl glycidate; ethyl-2-methyl butyrate; flor acetate; fructalate; frutene; gamma decalactone; geranyl acetate; habanolide; helvetolide; hexyl acetate; iso bornyl acetate; iso nonyl acetate; isoamyl butyrate; jasmal; methyl anthranilate; methyl benzoate; methyl dihydro jasmonate; methyl dioxolan; methyl laitone; methyl phenyl carbinyl acetate; musk RI; octahydro coumarin; phenoxy ethyl iso butyrate; phenyl ethyl tiglate; prenyl acetate; romandolide; sclareolate; terpinyl acetate; thesaron; triethyl citrate; undecalactone; verdox; violiff; zenolide; and combinations thereof.
G. The treatment composition according to any of paragraphs A-F, wherein the fragrance material comprises at least about 1%, by weight of the fragrance material, of aldehyde-containing PRMs, preferably at least about 5%, more preferably at least about 10%, by weight of the fragrance material, of aldehyde-containing PRMs.
H. The treatment composition according to any of paragraphs A-G, wherein the fragrance material further comprises no more than about 30%, by weight of the fragrance materials, of hydroxyl-containing PRMs, preferably from about 1% to about 30% of hydroxyl-containing PRMs.
I. The treatment composition according to any of paragraphs A-H, wherein the fragrance material comprises: from about 45% to about 55% of ester-containing PRMs; from about 10% to about 25% of aldehyde-containing PRMs; from about 5% to about 20% hydroxyl-containing PRMs; and optionally from about 1% to about 40% of additional PRMs.
J. The treatment composition according to any of paragraphs A-I, wherein the core further comprises a partitioning modifier, optionally present in the core at a level of from about 10% to about 50%, by weight of the core, preferably a partitioning modifier selected from the group consisting of vegetable oil, modified vegetable oil, mono-, di-, and tri-esters of C₄-C₂₄ fatty acids, isopropyl myristate, dodecanophenone, lauryl laurate, methyl behenate, methyl laurate, methyl palmitate, methyl stearate, and mixtures thereof, more preferably isopropyl myristate.
K. The treatment composition according to any of paragraphs A-J, wherein the biopolymer is selected from the group consisting of a polysaccharide, a protein, a nucleic acid, derivatives thereof, and combinations thereof; preferably wherein the biopolymer is selected from the group consisting of: chitosan, amine-modified starch, amine-modified dextran, amine-modified maltodextrin, amine-modified dextrin, amine-modified cellulose, amine-modified hemicellulose, chitin, amine-modified alginate, amine-modified lignin, amine-modified gum, amine-modified pectin, amine modified agar, melanin, silk fibronin, gelatin, collagen, casein, sericin, fibroin, whey protein, pea protein, zein, soy protein, plant storage protein, gluten, peptide, actin, polynucleotides, RNA, DNA, derivatives thereof, and combinations thereof.
L. The treatment composition according to any of paragraphs A-K, wherein the biopolymer is chitosan, a derivative thereof, or a combination thereof, preferably wherein the biopolymer is acid-treated chitosan.
M. The treatment composition according to any of paragraphs A-L, wherein the biopolymer, preferably chitosan, more preferably acid-treated chitosan, is characterized by a molecular weight of from about 1 kDa to about 1000 kDa, preferably from about 50 kDa to about 600 kDa, more preferably from about 100 kDa to about 500 kDa, even more preferably from about 100 kDa to about 300 kDa, even more preferably from about 100 kDa to about 200 kDa.
N. The treatment composition according to any of paragraphs A-M, wherein the cross-linking agent is a material selected from the group consisting of a polyisocyanate, a polyacrylate, a poly(meth)acrylate, a polyisothiocyanate, an aldehyde, an epoxy compound, a polyphenol, a carbonyl halide, an aziridine, and combinations thereof, preferably selected from the group consisting of a polyisocyanate, an epoxy compound, a bifunctional aldehyde, and combinations thereof, more preferably a polyisocyanate, even more preferably a polyisocyanate selected from the group consisting of: a polyisocyanurate of toluene diisocyanate; a trimethylol propane adduct of toluene diisocyanate; a trimethylol propane adduct of xylylene diisocyanate; 2,2′-methylenediphenyl diisocyanate; 4,4′-methylenediphenyl diisocyanate; 2,4′-methylenediphenyl diisocyanate; [diisocyanato(phenyl)methyl]benzene; toluene diisocyanate; tetramethylxylidene diisocyanate; naphthalene-1,5-diisocyanate; 1,4-phenylene diisocyanate; 1,3-diisocyanatobenzene; derivatives thereof (such as pre-polymers, oligomers, and/or polymers thereof); and combinations thereof.
O. The treatment composition according to any of paragraphs A-N, wherein the delivery particles are characterized by a volume-weighted median particle size from about 1 to about 100 microns, preferably from about 10 to about 100 microns, preferably from about 15 to about 50 microns, more preferably from about 20 to about 40 microns, even more preferably from about 20 to about 30 microns.
P. The treatment composition according to any of paragraphs A-O, wherein the shells of the delivery particles degrade at least 60% in 60 days when tested according to test method OECD 301B.
Q. The treatment composition according to any of paragraphs A-P, wherein the treatment adjunct is selected from the group consisting of surfactants, conditioning actives, deposition aids, rheology modifiers or structurants, bleach systems, stabilizers, builders, chelating agents, dye transfer inhibiting agents, dispersants, enzymes, enzyme stabilizers, catalytic metal complexes, polymeric dispersing agents, clay and soil removal/anti-redeposition agents, brighteners, suds suppressors, silicones, hueing agents, aesthetic dyes, neat perfume, additional perfume delivery systems, structure elasticizing agents, carriers, hydrotropes, processing aids, anti-agglomeration agents, coatings, formaldehyde scavengers, pigments, and mixtures thereof.
R. The treatment composition according to any of paragraphs A-Q, wherein the treatment adjunct comprises anionic surfactant, a cationic conditioning agent, or a mixture thereof.
S. The treatment composition according to any of paragraphs A-R, wherein the treatment composition is a fabric care composition, a hard surface cleaner composition, a dish care composition, a hair care composition, a body cleansing composition, or a mixture thereof, preferably a fabric care composition, more preferably a fabric care composition that is a laundry detergent composition, a fabric conditioning composition, a laundry additive, a fabric pre-treat composition, a fabric refresher composition, or a mixture thereof.
T. The treatment composition according to any of paragraphs A-S, wherein the treatment composition is in the form of a liquid composition, a granular composition, a hydrocolloid, a single-compartment pouch, a multi-compartment pouch, a dissolvable sheet, a pastille or bead, a fibrous article, a tablet, a stick, a bar, a flake, a foam/mousse, a non-woven sheet, or a mixture thereof, preferably a liquid composition.
U. The treatment composition according to any of paragraphs A-T, wherein the consumer product composition further comprises from about 50% to about 99%, by weight of the treatment composition, of water, preferably from about 60% to about 98%, more preferably from about 80% to about 96%, by weight of the consumer product composition, of water.
V. A method of treating a surface, the method comprising the step of: contacting the surface, preferably a fabric, with a treatment composition according to any of paragraphs A-U.
W. A method of making a treatment composition, the method comprising the steps of: providing a base composition, wherein the base composition comprises a treatment adjunct, and combining a population of delivery particles with the base composition, wherein the delivery particles comprise a core and a shell surrounding the core, wherein the shell comprises a polymeric material, wherein the polymeric material comprises a reaction product of a biopolymer and a cross-linking agent, wherein the biopolymer comprises primary amine groups, wherein the core comprises a fragrance material, wherein the fragrance material comprises: (a) at least about 30%, by weight of the fragrance material, of ester-containing perfume raw materials (“PRMs”), and (b) at least about 0.5wt %, by weight of the fragrance material, aldehyde-containing PRMs.

### TEST METHODS

It is understood that the test methods disclosed in the Test Methods section of the present application should be used to determine the respective values of the parameters of Applicant's claimed subject matter as claimed and described herein.

### Method to Determine S-ESTER Value

The S-ESTER values are based on the atom level electrotopological state (E-State) indices of one or more compounds. E-State values are molecular descriptors that can be found in literature and/or determined using commercially available software programs. The E-State values effectively look at each atom in a molecule and reflect the valence state electronegativity of the atom, which is affected by atoms connected to it. Electropological State indices are discussed more in Hall et al., Electrotopological State Indices for Atom Types: A Novel Combination of Electronic, Topological, and Valence State Information, J. Chem. Inf. Comput. Sci. 1995, 35, 1039-1045.

To determine E-State and S-ESTER values for the purposes described in the present disclosure, E-State values are computed using software program winMolconn version 1.2.2.3 (available from Hall Associates Consulting of Quincy, MA), used according to the manufacturer's instructions. Structures are prepared using a 2D connection table (SDF format or SMILES). The value labels used in the model test method computations are the same labels reported by the winMolconn; their descriptions and definitions can be found listed in the winMolconn documentation associated with the software.

To determine the S-ESTER value for a particular perfume raw material or other compound, the E-State values for each atom are computed using software program winMolconn version 1.2.2.3. The S-ESTER value is the sum of the atom level electrotopological state (E-State) values for all carbonyl carbons and oxygens in ester groups in the molecule. If a compound includes multiple ester groups, the E-State values for all of the ester groups are added. If a compound has no ester groups, the S-ESTER value of the compound is zero.

As an example, the table below shows the SMILES input for an ester-containing PRM, flor acetate, as well as the resulting S-ESTER value provided by the software.

| SMILES input in winMolconn software | Nme | SEster |
|---|---|---|
| CC(OC1C2C3C(C═CC3)C(C2)C1)═O | flor | 16.2105 |
| | acetate | |

To determine the S-ESTER value for a mixture of compounds, such as a fragrance material that comprises more than one perfume raw material, the S-ESTER values for each compound is first determined as described above. The S-ESTER value of the mixture is calculated by finding a weighted average of S-ESTER values, based on the relative proportions (by weight percent) of each individual compound in the mixture.

### Determination of a Polymer's Molecular Weight and Related Parameters

The following method describing gel permeation chromatograph with multi-angle light scatter and refractive index detection (GPC-MALS/RI) is used to find molecular weight distribution measurements and related values of the polymers described herein.

Gel Permeation Chromatography (GPC) with Multi-Angle Light Scattering (MALS) and Refractive Index (RI) Detection (GPC-MALS/RI) permits the measurement of absolute molecular weight of a polymer without the need for column calibration methods or standards. The GPC system allows molecules to be separated as a function of their molecular size. MALS and RI allow information to be obtained on the number average (Mn) and weight average (Mw) molecular weight.

The Mw distribution of water-soluble polymers like chitosan is typically measured by using a Liquid Chromatography system (e.g., Agilent 1260 Infinity pump system with OpenLab Chemstation software, Agilent Technology, Santa Clara, CA, USA) and a column set (e.g., 2 Tosoh TSKgel G6000WP 7.8×300 mm 13 um pore size, guard column A0022 6 mm×40 mm PW x1-cp, King of Prussia, PA) which is operated at 40° C. The mobile phase is 0.1M sodium nitrate in water containing 0.02% sodium azide and 0.2% acetic acid. The mobile phase solvent is pumped at a flow rate of 1 mL/min, isocratically. A multiangle light scattering (18-Angle MALS) detector DAWN® and a differential refractive index (RI) detector (Wyatt Technology of Santa Barbara, Calif., USA) controlled by Wyatt Astra® software v8.0 are used.

A sample is typically prepared by dissolving chitosan materials in the mobile phase at ˜1 mg per ml and by mixing the solution for overnight hydration at room temperature. The sample is filtered through a 0.8 μm Versapor membrane filter (PALL, Life Sciences, NY, USA) into the LC autosampler vial using a 3-ml syringe before the GPC analysis.

A dn/dc value (differential change of refractive index with concentration, 0.15) is used for the number average molecular weight (Mn), weight average molecular weight (Mw), Z-average molecular weight (Mz), molecular weight of the peak maxima (Mp), and polydispersity (Mw/Mn) determination by the Astra detector software.

### Viscosity

Viscosity of liquid finished product is measured using an AR 550 rheometer/viscometer from TA instruments (New Castle, DE, USA), using parallel steel plates of 40 mm diameter and a gap size of 500 μm. The high shear viscosity at 20 s⁻¹ and low shear viscosity at 0.05 s⁻¹ is obtained from a logarithmic shear rate sweep from 0.01 s⁻¹ to 25 s⁻¹ in 3 minutes time at 21° C.

### Test Method for Determining log P

The value of the log of the Octanol/Water Partition Coefficient (log P) is computed for each material (e.g., each PRM in the perfume mixture) being tested. The log P of an individual material (e.g., a PRM) is calculated using the Consensus log P Computational Model, version 14.02 (Linux) available from Advanced Chemistry Development Inc. (ACD/Labs) (Toronto, Canada) to provide the unitless log P value. The ACD/Labs' Consensus log P Computational Model is part of the ACD/Labs model suite.

### Volume-Weighted Particle Size and Size Distribution

The volume-weighted particle size distribution is determined via single-particle optical sensing (SPOS), also called optical particle counting (OPC), using the AccuSizer 780 AD instrument and the accompanying software CW788 version 1.82 (Particle Sizing Systems, Santa Barbara, California, U.S.A.), or equivalent. The instrument is configured with the following conditions and selections: Flow Rate=1 ml/sec; Lower Size Threshold=0.50 μm; Sensor Model Number=Sensor Model Number=LE400-05 or equivalent; Autodilution=On; Collection time=60 sec; Number channels=512; Vessel fluid volume=50 ml; Max coincidence=9200. The measurement is initiated by putting the sensor into a cold state by flushing with water until background counts are less than 100. A sample of delivery capsules in suspension is introduced, and its density of capsules adjusted with DI water as necessary via autodilution to result in capsule counts of at least 9200 per ml. During a time period of 60 seconds the suspension is analyzed. The resulting volume-weighted PSD data are plotted and recorded, and the values of the desired volume-weighted particle size (e.g., the median/50^{th} percentile, 5^{th} percentile, and/or 90^{th} percentile) are determined.

### Procedure for Determination of % Degradation

To determine % degradation, the procedure set forth in the “OECD Guideline for Testing of Chemicals” 301B CO₂ Evolution (Modified Sturm Test), adopted 17 Jul. 1992, is used. For ease of reference, this test method is referred to herein as test method OECD 301B.

### Fabric Treatment

Miele washing machines were used to treat the fabrics. For each treatment, the washing machine was loaded with 3 kg fabric, comprising 1100 g knitted cotton fabric, 1100 g polyester-cotton fabrics (50/50). Also 18 terry towel cotton tracers are added, which weigh together about 780 g. Prior to the treatment, this load was preconditioned twice with 79 g IEC A Base detergent, which is unperfumed and supplied by WFK Testgewebe GmbH, using the 95° C. short cotton cycle followed by two additional 95° C. washes without detergent.

Prior to the test treatment, the load is preconditioned twice, each time using the 95° C. short cotton cycle with 79 g of unperfumed IEC A Base detergent (ex WFK Testgewebe GmbH), followed by two additional 95° C. washes without detergent.

For the test treatment, the load is washed using a 40° C. short cotton cycle, 1200 rpm spin speed with 79 g IEC A Base detergent, which is added at the start of the wash cycle in the appropriate dispenser. A dosage of 35 g of test fabric treatment composition (e.g., according to Examples) is added in the appropriate dispenser. At the end of the wash cycle, the terry towel tracers are removed from the washing machine and line dried overnight. The next day, expert perfumers perform an olfactive assessment for perfume intensity on the dry terry towel tracers. For comparative purposes a reference treatment is also executed where the same fragrance is used as in the test sample but using polyacrylate capsule as delivery particle. All comparative treatments are washed at the same day and analyzed on the same day

### Method of Olfactive Evaluation

After the fabrics have been treated, expert perfumers perform an olfactive assessment for on the dry fabrics perfume intensity at the DRY touchpoints (Dry Fabric Odor=DFO) and the scores are averaged. Scores are based on a perfume odor intensity scale from 0 to 100, where 0=no perfume odor, 25=slight perfume odor, 50=moderate perfume odor, 75=strong perfume odor, and 100=extremely strong perfume odor.

### Method to Determine Headspace Concentration Above Treated Dry Fabrics

The cotton tracers are analyzed by a fast headspace GC/MS (gas chromatography mass spectrometry) approach. 4×4 cm aliquots of the terry towel cotton tracers were transferred to 25 ml headspace vials. The fabric samples were equilibrated for 10 minutes @65° C. The headspace above the fabrics was sampled via SPME (50/30 μm DVB/Carboxen/PDMS) approach for 5 minutes. The SPME fiber was subsequently on-line thermally desorbed into the GC. The analytes were analyzed by fast GC/MS in full scan mode. Ion extraction of the specific masses of the PRMs was used to calculate the total HS response and perfume headspace composition above the tested legs.

### Total wt % PRM Degraded in Delivery Particles Composition

To determine the total amount of degraded PRMs in the delivery particle composition, total fragrance (encapsulated+non-encapsulated) is measured using the fragrance raw material as reference. For this purpose, Gas Chromatography with Mass Spectroscopy Detector (GC-MS) is employed. Suitable equipment includes: Agilent GC8890 equipped with Agilent 5977B mass spectrometer or equivalent, capillary column operation, quantitation based on extracted ion capability, autosampler; and 30m×0.25mm nominal diameter column, 1 μm film thickness, J&W 122-5533 DB-5MS, or equivalent.

Approximately 0.1 g of the delivery particles composition, is weighed and the weight recorded, then an internal standard (Tonalid, ex Merck) and 10 mL ethanol are added to the sample. The suspension is heated at 60° C. for 45 minutes, and is then placed in an ultrasonic bath (like Branson 3510) for 15 minutes. Then, the cooled solution is filtered through 0.45 μm pore size PTFE syringe filter and analyzed via GC-MS. The amount of PRMs measured is determined from the response of each PRM and the total wt % PRM degradation can be determined as follows:

$$Total wt \% PRM degraded = \frac{initial fragrance - {\sum }_{i}^{n} PRM measured}{initial fragrance} x 100$$

wherein i is each single PRM that is part of the fragrance composition and n the total number of PRMs in the fragrance composition.

### EXAMPLES

The examples provided below are intended to be illustrative in nature and are not intended to be limiting.

### Example 1. Exemplary Fragrance Material

Table 1 shows an exemplary fragrance material according to the present disclosure. The perfume raw materials are listed, along with relative weight percentages (by weight of the fragrance material) and the S-ESTER values for each perfume raw materials. From these values, the S-ESTER value of the total fragrance material can be calculated as a weight-averaged S-ESTER value.

TABLE 1

| | | | S-ESTER | |
|---|---|---|---|---|
| | | | value | Weighted S- |
| Name PRM | CAS | % Weight | (indiv. PRM) | ESTER value |
| Flor Acetate ^{E} | 5413-60-5 | 15 | 16.2105 | 2.432 |
| Propyl cinnamate ^{E} | 7778-83-8 | 10 | 15.6716 | 1.567 |
| Melonal ^{A} | 106-72-9 | 15 | 0 | 0 |
| Ethyl vanillin ^{A} | 121-32-4 | 8 | 0 | 0 |
| Fructalate ^{E} | 72903-27-6 | 12 | 32.6217 | 3.915 |
| beta-Ionone | 14901-07-6 | 10 | 0 | 0 |
| Triplal ^{A} | 67801-65-4 | 14 | 0 | 0 |
| (+)-Citronellol | 1117-61-9 | 16 | 0 | 0 |

| | |
|---|---|
| Weight averaged S-ESTER value for the total fragrance material = | 7.913 |

^{E} = material contains an ester functional group
^{A} = material contains an aldehyde functional group

Based on the information in Table 1, the exemplary fragrance material includes, by weight of the fragrance material, 37% of ester-containing materials and 37% of aldehyde-containing materials.

Additionally, the S-ESTER value of the total fragrance material is determined to be 7.913, calculated according to the weighted average of the S-ESTER values of the PRMs.

### Example 2. Illustrative Delivery Particle Synthesis

In the following example, the abbreviations correspond to the materials listed in Table 2.

TABLE 2

| Trade Name | Company/City | Material |
|---|---|---|
| ChitoClear | Primex EHF, Siglufjordur, | Chitosan |
| (grade: 44030- | Iceland | |
| fg95ULV) | | |
| Takenate D-110N | Mitsui Chemicals America, Inc., | Polyisocyanate |
| | Rye Brook, NY | prepolymer |

A water phase is prepared by dispersing 92.19 g ChitoClear into 1956.6 g water while mixing in a jacketed reactor. The pH of the water phase is then adjusted to between 5.0 to 5.3 using concentrated HCl under agitation. The water phase temperature is then increased to 85° C. over 60 minutes and then held at 85° C. for 2 hours to acid-treat the ChitoClear. The water phase temperature is then reduced to 25° C. after the hydrolyzing step over a period of 90 minutes. An oil phase is prepared by mixing 716.37 g perfume oil according to the present disclosure (e.g., the fragrance material of Example 1) and 179.11 g isopropyl myristate together along with 19.54 g Takenate D-110N at room temperature. The oil phase is added to the water phase under high shear milling to obtain an emulsion. The emulsion is heated to 40° C. over 30 minutes and held for 60 minutes. The emulsion is then heated to 85° C. and maintained at this temperature for 6 hours while mixing.

### Example 3. Effect of Encapsulated Perfume Selection

To compare different chitosan delivery particles, samples of liquid fabric enhancers (“LFE”) are prepared with the different particles. The same perfume is used in delivery particles assessed in reference treatments, and each core also includes approximately 20%-35% of partitioning modifier (i.e., isopropyl myristate). The LFE compositions include a softening active (diester quat) present at 6%, the relevant delivery particles added at a level sufficient to provide 0.2% encapsulated fragrance, 0.11% structurant (cationic polymer—Flosoft FS222, ex SNF), and various processing minors, with pH adjusted to be approximately 3.

For the examples, liquid fabric enhancer products having the general formula according to Table 3 below are prepared.

TABLE 3

| | % Active |
|---|---|
| Ingredient | (w/w) |
| DiEster Quat | 6% |
| Encapsulated Perfume (by weight of fragrance | 0.2% |
| delivered) | |
| Formic Acid | 0.045% |
| Hydrochloric acid | 0.0075% |
| Sodium Hydroxyethane diphosphonic acid | 0.0071% |
| Structurant (cationic polymer) | 0.11% |
| Antifoam (silicone) | 0.004% |
| Water | Balance |

Seven different fragrances are encapsulated in both delivery particles according to the present disclosure (e.g., walls made from chitosan and cross-linked with isocyanate) and reference polyacrylate (“PAC”) delivery particles. The compositions of the tested fragrances are generally described below in Table 3; to note, perfume raw materials that have more than one type of functional moiety are counted in each “functionality” category, which may result in a PRM being counted more than once and the percentages seeming to add up to more than 100%. The S-ESTER values for each fragrance are also provided in Table 4. Fragrances 1-4 (“Fragr. 1,” etc.) are fragrance materials according to the present disclosure; Fragrances 5-7 (“Fragr. 5,” etc.) are comparative fragrance materials.

The walls of the delivery particles of the present disclosure used in the experiment are made from an amine-containing biopolymer (acid-treated chitosan) and a cross-linking agent (polyisocyanate) (target volume-weighted median particle size=28 microns). The reference polyacrylate delivery particles are made substantially according to the methods described in US Publication 2011/0268802. Notably, the walls of the PAC delivery particles are not made from monomers that have primary amines, so reactivity with aldehydic PRMs is not significantly expected. The reference PAC particles for Fragrances 1-5 are characterized by a core:shell weight ratio of about 90:10. The reference PAC particles for Fragrances 6 and 7 are characterized by a core:shell weight ratio of from about 97:3 to about 98:2.

The composition of the fragrance in the delivery particles are analyzed using the analytical filtration method as described in the Test Method section above and compared to the fragrance oil prior to encapsulation. From this analysis, the total wt % PRM degraded is calculated according to the method described above.

The results are provided below in Table 4. For comparative purpose, olfactive DFO data for the chitosan-based delivery particle are reported as a Delta value versus the DFO resulting from the reference polyacrylate delivery particle that includes the same fragrance, according to the equation below.

Delta DFO=DFO Chitosan delivery particle (test)−DFO PAC delivery particle (ref)

Delta DFO entries with an asterisk (*) are statistically significant compared the ref value.

TABLE 4

| | | | | | Fragr. 5 | Fragr. 6 | Fragr. 7 |
|---|---|---|---|---|---|---|---|
| | Fragr. 1 | Fragr. 2 | Fragr. 3 | Fragr. 4 | (comp.) | (comp.) | (comp.) |
| % Ester | 39.3 | 52.4 | 52.7 | 54.3 | 20.9 | 25.7 | 27 |
| % Aldehyde | 15 | 17 | 3.85 | 10.4 | 33.5 | 19.8 | 15.5 |
| % Alcohol | 13.1 | 5 | 11.4 | 19.9 | 17.5 | 17.5 | 18.4 |
| % Ketone | 18.7 | 4.2 | 24 | 6.93 | 1.7 | 10.6 | 11.2 |
| S-ESTER | 5.62 | 7.47 | 8.65 | 8.8 | 3.31 | 4.11 | 4.32 |
| Value of | | | | | | | |
| Fragrance | | | | | | | |
| Olfactive | 0.1 | 0 | −2.5 | 2.7 | −18.6* | −13.2* | −11.6* |
| Score | | | | | | | |
| (Delta DFO | | | | | | | |
| vs. ref) | | | | | | | |
| Total wt % | 4.9 | 2.2 | 3.6 | 3.2 | 15.6 | 15.3 | 8.7 |
| PRM | | | | | | | |
| degraded | | | | | | | |

As shown in Table 4, the inventive delivery particles, which are characterized by a core that includes a fragrance with greater than 30% esters and/or an S-ESTER value of greater than 5, perform substantially equally on an olfactory basis compared to the reference polyacrylate capsules. Further, they show total % degradation of PRMs in the core of less than 5%. On the other hand, the comparative delivery particles, which have similar walls but are characterized by a core that includes a fragrance with less than 30% esters and/or an S-ESTER value of less than 5, perform significantly worse compared to the reference polyacrylate capsules. Further, they show total % degradation of PRMs in the core of greater than 5%.

The dimensions and values disclosed herein are not to be understood as being strictly limited to the exact numerical values recited. Instead, unless otherwise specified, each such dimension is intended to mean both the recited value and a functionally equivalent range surrounding that value. For example, a dimension disclosed as “40 mm” is intended to mean “about 40 mm.”

Every document cited herein, including any cross referenced or related patent or application and any patent application or patent to which this application claims priority or benefit thereof, is hereby incorporated herein by reference in its entirety unless expressly excluded or otherwise limited. The citation of any document is not an admission that it is prior art with respect to any invention disclosed or claimed herein or that it alone, or in any combination with any other reference or references, teaches, suggests or discloses any such invention. Further, to the extent that any meaning or definition of a term in this document conflicts with any meaning or definition of the same term in a document incorporated by reference, the meaning or definition assigned to that term in this document shall govern.

While particular embodiments of the present invention have been illustrated and described, it would be obvious to those skilled in the art that various other changes and modifications can be made without departing from the spirit and scope of the invention. It is therefore intended to cover in the appended claims all such changes and modifications that are within the scope of this invention.

What is claimed is:

1. A treatment composition comprising:
a treatment adjunct, and
a population of delivery particles,
wherein the delivery particles comprise a core and a shell surrounding the core,
wherein the shell comprises a polymeric material,
wherein the polymeric material comprises a reaction product of a biopolymer and a cross-linking agent,
wherein the biopolymer comprises primary amine groups,
wherein the core comprises a fragrance material, wherein the fragrance material comprises:
(a) at least about 30%, by weight of the fragrance material, of ester-containing perfume raw materials (“PRMs”), and
(b) at least about 0.5wt %, by weight of the fragrance material, aldehyde-containing PRMs.

2. The treatment composition according to claim 1, wherein the fragrance material comprises at least about 35%, by weight of the fragrance material, of ester-containing PRMs.

3. The treatment composition according to claim 1, wherein the ester-containing perfume raw materials comprise one or more materials selected from the group consisting of: 4-tertiary butyl cyclohexyl acetate; allyl amyl glycolate; allyl caproate; allyl cyclohexane propionate; allyl heptoate; amyl-acetate (isomer blends); Cis-3-hexenyl acetate; cis-3-hexenyl salicylate; cyclobute; cyclo galbanate; cyclobutanate; dihydro terpinyl acetate; dimethyl benzyl carbinyl acetate; dimethyl benzyl carbinyl butyrate; exaltolide total; ethyl 2 methyl pentanoate; ethyl acetate; ethyl butyrate; ethylene brassylate; ethyl isovalerate; ethyl laitone; ethyl methyl phenyl glycidate; ethyl methyl phenyl glycidate; ethyl-2-methyl butyrate; flor acetate; fructalate; frutene; gamma decalactone; geranyl acetate; habanolide; helvetolide; hexyl acetate; iso bornyl acetate; iso nonyl acetate; isoamyl butyrate; jasmal; methyl anthranilate; methyl benzoate; methyl dihydro jasmonate; methyl dioxolan; methyl laitone; methyl phenyl carbinyl acetate; musk RI; octahydro coumarin; phenoxy ethyl iso butyrate; phenyl ethyl tiglate; prenyl acetate; romandolide; sclareolate; terpinyl acetate; thesaron; triethyl citrate; undecalactone; verdox; violiff; zenolide; and combinations thereof.

4. The treatment composition according to claim 1, wherein the fragrance material comprises at least about 1%, by weight of the fragrance material, of aldehyde-containing PRMs.

5. The treatment composition according to claim 1, wherein the fragrance material further comprises no more than about 30%, by weight of the fragrance materials, of hydroxyl-containing PRMs.

6. The treatment composition according to claim 1, wherein the fragrance material comprises:
from about 45% to about 55% of ester-containing PRMs;
from about 10% to about 25% of aldehyde-containing PRMs;
from about 5% to about 20% hydroxyl-containing PRMs.

7. The treatment composition according to claim 1, wherein the core further comprises a partitioning modifier.

8. The treatment composition according to claim 1, wherein the biopolymer is selected from the group consisting of a polysaccharide, a protein, a nucleic acid, derivatives thereof, and combinations thereof.

9. The treatment composition according to claim 1, wherein the biopolymer is chitosan, a derivative thereof, or a combination thereof.

10. The treatment composition according to claim 1, wherein the biopolymer is characterized by a molecular weight of from about 1 kDa to about 1000 kDa.

11. The treatment composition according to claim 1, wherein the cross-linking agent is a material selected from the group consisting of a polyisocyanate, a polyacrylate, a poly(meth)acrylate, a polyisothiocyanate, an aldehyde, an epoxy compound, a polyphenol, a carbonyl halide, an aziridine, and combinations thereof.

12. The treatment composition according to claim 1, wherein the delivery particles are characterized by a volume-weighted median particle size from about 1 to about 100 microns.

13. The treatment composition according to claim 1, wherein the shells of the delivery particles degrade at least 60% in 60 days when tested according to test method OECD 301B.

14. The treatment composition according to claim 1, wherein the treatment adjunct is selected from the group consisting of surfactants, conditioning actives, deposition aids, rheology modifiers or structurants, bleach systems, stabilizers, builders, chelating agents, dye transfer inhibiting agents, dispersants, enzymes, enzyme stabilizers, catalytic metal complexes, polymeric dispersing agents, clay and soil removal/anti-redeposition agents, brighteners, suds suppressors, silicones, hueing agents, aesthetic dyes, neat perfume, additional perfume delivery systems, structure elasticizing agents, carriers, hydrotropes, processing aids, anti-agglomeration agents, coatings, formaldehyde scavengers, pigments, and mixtures thereof.

15. The treatment composition according to claim 1, wherein the treatment adjunct comprises anionic surfactant, a cationic conditioning agent, or a mixture thereof.

16. The treatment composition according to claim 1, wherein the treatment composition is a fabric care composition, a hard surface cleaner composition, a dish care composition, a hair care composition, a body cleansing composition, or a mixture thereof.

17. The treatment composition according to claim 1, wherein the treatment composition is in the form of a liquid composition, a granular composition, a hydrocolloid, a single-compartment pouch, a multi-compartment pouch, a dissolvable sheet, a pastille or bead, a fibrous article, a tablet, a stick, a bar, a flake, a foam/mousse, a non-woven sheet, or a mixture thereof.

18. A treatment composition comprising:
a treatment adjunct, and
a population of delivery particles,
wherein the delivery particles comprise a core and a shell surrounding the core,
wherein the shell comprises a polymeric material,
wherein the polymeric material comprises reaction product of a biopolymer and a cross-linking agent,
wherein the biopolymer comprises primary amine groups,
wherein the core comprises a fragrance material,
wherein the fragrance material comprises one or more perfume raw materials (“PRMs”), and
wherein the fragrance material is characterized by one or more of the following:
(a) as having an S-ESTER value of at least about 5, wherein the S-ESTER value of the fragrance material is calculated as the weight average S-ESTER values of the perfume raw materials; and/or
(b) as comprising at least about 30%, by weight of the fragrance materials, of perfume raw materials having S-ESTER values of 13 or greater.

19. The treatment composition according to claim 18, wherein the fragrance material is characterized as having an S-ESTER value of at least about 5.5.

20. The treatment composition according to claim 18, wherein the fragrance material comprises at least about 40% of perfume raw materials having S-ESTER values of about 13 or greater.
